(19) United States
(12) Patent Application Publication
(10) Pub. No.: US 2022/0380348 A1
(43) Pub. Date: Dec. 1, 2022

(54) AXL INHIBITOR FORMULATIONS

(71) Applicant: Sumitomo Dainippon Pharma Oncology, Inc., Cambridge, MA (US)

(72) Inventors: Adam Siddiqui-Jain, South Jordan, UT (US); Steven L. Warner, Ph.D., Sandy, UT (US); Paul Flynn, Citrus Heights, CA (US); Akihito NONOYAMA, Osaka-shi, Osaka (JP); Akihito Kiguchiya, Osaka-shi, Osaka (JP)

(21) Appl. No.: 17/761,993

(22) PCT Filed: Oct. 2, 2020
(86) PCT No.: PCT/US2020/054032

§ 371 Date: Mar. 18, 2022

Related U.S. Application Data
(60) Provisional application No. 62/910,709, filed on Oct. 4, 2019.

(51) Int. Cl. C07D 403/12; A61K 9/48

(52) U.S. Cl. CPC C07D 403/12; A61K 9/4858; C07B 2200/13

(57) ABSTRACT

Formulations of Compound 1 or a pharmaceutically acceptable salt thereof are described. Also disclosed are capsules having a dry blended powder comprising Compound 1 or a pharmaceutically acceptable salt thereof. The Compound 1 or a pharmaceutically acceptable salt thereof may be a tartrate salt of Compound 1, such as a mono-, sub-, or di-tartrate salt, and including crystalline forms thereof. The formulations may include excipients such as diluents (e.g., microcrystalline cellulose, lactose); disintegrants (e.g., croscarmellose sodium); and lubricants (e.g., magnesium stearate).

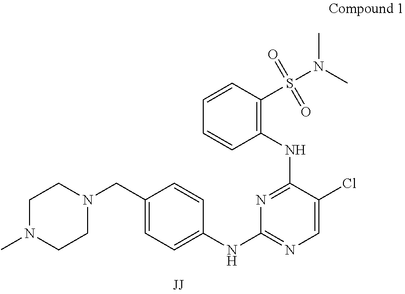

### TECHNICAL FIELD

The present disclosure relates to formulations of Compound 1 or a pharmaceutically acceptable salt thereof.

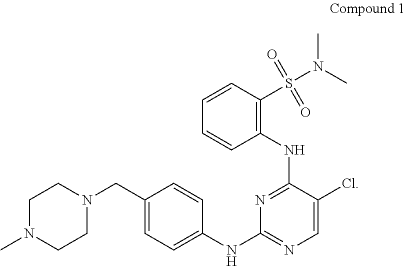

### BACKGROUND

AXL is a cell surface receptor tyrosine kinase of the TAM family. The AXL receptor binds growth factors like vitamin K-dependent protein growth-arrest-specific gene 6 (GAS6) and transduces signals from the extracellular matrix into the cytoplasm. It is reported that AXL is an inhibitor of the innate immune response and may play a role in multiple cellular processes relating to cell growth and development.

AXL is found to be involved in various aspects of tumor growth, including cancer cell proliferation, invasiveness and migration, as well as stemness, angiogenesis, and immune modulation. As such, AXL becomes a promising cancer treatment target.

### SUMMARY

In one aspect, the present disclosure provides a formulation comprising Compound 1 or a pharmaceutically acceptable salt thereof and one or more pharmaceutically acceptable excipients.

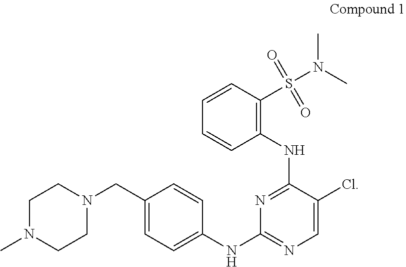

In another aspect, the present disclosure provides a capsule comprising Compound 1 or a pharmaceutically acceptable salt thereof and one or more pharmaceutically acceptable excipients.

In another aspect, the present disclosure provides a process for preparing a formulation (e.g., a capsule) comprising Compound 1 or a pharmaceutically acceptable salt thereof and one or more pharmaceutically acceptable excipients.

In some embodiments, the one or more pharmaceutically acceptable excipients includes microcrystalline cellulose, lactose (e.g., lactose monohydrate), croscarmellose sodium, magnesium stearate, or any combination thereof.

In some embodiments, the Compound 1 or a pharmaceutically acceptable salt thereof comprises a tartrate salt of Compound 1. In some embodiments, the tartrate salt of Compound 1 is a di-tartrate salt of Compound 1. In some embodiments, the di-tartrate salt of Compound 1 is the crystalline Form A di-tartrate salt of Compound 1.

These and other objects, features, and advantages of the disclosure will become apparent from the following detailed description of the various aspects and embodiments of the disclosures taken in conjunction with the accompanying tables and drawings.

All published documents cited herein are hereby incorporated herein by reference in their entirety.

### BRIEF DESCRIPTION OF THE DRAWINGS

FIG. 1 is a flow chart of an exemplary manufacturing process.

FIG. 2A illustrates an x-ray diffractogram obtained from XRPD analysis for crystalline Form A.

FIG. 2B illustrates a ¹HNMR spectrum of crystalline Form A.

FIG. 2C illustrates a ¹HNMR spectrum of crystalline Form A′.

FIG. 3A illustrates an x-ray diffractogram obtained from XRPD analysis for crystalline Form B.

FIG. 3B illustrates a ¹HNMR spectrum of crystalline Form B.

FIG. 4A illustrates an x-ray diffractogram obtained from XRPD analysis for polymorph Form D.

FIG. 4B illustrates a ¹HNMR spectrum of crystalline Form D.

FIG. 5 shows a TGA and DSC plot obtained for crystalline Form A.

FIG. 6 shows a comparison between Form B (upper) and Form A (lower).

FIG. 7 shows XRPD peaks characteristic of Form B.

FIG. 8 shows TGA/DSC curves of Form B.

FIG. 9 shows XRPD peaks characteristic of Form D.

FIG. 10 shows TGA/DSC curves of Form D.

FIG. 11A shows a comparison between XRPD diffractograms of crystalline Forms A, B, and C.

FIG. 11B shows a comparison between XRPD diffractograms of crystalline Forms D, E, F, G, H, and I.

FIGS. 12A-12I show thermal behavior (DSC/TGA charts) for crystalline Forms A through I.

FIG. 13 shows moisture sorption isotherms for Forms A′, A, B, C, and D.

FIG. 14 shows the comparison of XRPD patterns of Form A and Form D between before and after moisture sorption isotherm.

FIG. 15 shows Raman spectra and its PCA data for Form A and Form B.

FIGS. 16A-16D show the ¹HNMR spectrum for the reaction products of Example 7.

### DETAILED DESCRIPTION

### I. Definitions

Unless defined otherwise, all technical and scientific terms used herein have the same meaning as commonly understood by one of ordinary skill in the art to which this disclosure belongs. Accordingly, the following terms are intended to have the following meanings:

As used herein, the singular forms “a”, “an” and “the” are intended to include the plural forms as well, unless the context clearly indicates otherwise.

Unless otherwise specified, the word “includes” (or any variation thereon, e.g., “include”, “including”, etc.) is intended to be open-ended. For example, “A includes 1, 2 and 3” means that A includes but is not limited to 1, 2 and 3.

Unless the context requires otherwise, throughout the present specification and claims, the word “comprise” and variations thereof, such as, “comprises” and “comprising” are to be construed in an open, inclusive sense (i.e., as “including, but not limited to”).

“Pharmaceutically acceptable” or “physiologically acceptable” refer to compounds, salts, compositions, dosage forms and other materials which are useful in preparing a formulation that is suitable for veterinary or human pharmaceutical use.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid (e.g., L-(+)-tartaric acid), thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2 dimethylaminoethanol, 2 diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N ethylpiperidine, polyamine resins and the like. Particularly preferred organic bases are isopropylamine, diethylamine, ethanolamine, trimethylamine, dicyclohexylamine, choline and caffeine.

In some embodiments, pharmaceutically acceptable salts include quaternary ammonium salts such as quaternary amine alkyl halide salts (e.g., methyl bromide).

“About” and “approximately,” when used in connection with a numeric value or range of values which is provided to describe a particular solid form, e.g., a specific temperature or temperature range, such as, for example, that describing a melting, dehydration, desolvation or glass transition; a mass change, such as, for example, a mass change as a function of temperature or humidity; a solvent or water content, in terms of, for example, mass or a percentage; or a peak position, such as, for example, in analysis by IR or Raman spectroscopy or XRPD; indicate that the value or range of values may deviate to an extent deemed reasonable to one of ordinary skill in the art while still describing the particular solid state form. Specifically, the terms “about” and “approximately,” when used in this context, indicate that the numeric value or range of values may vary by 20%, 10%, 9%, 8%, 7%, 6%, 5%, 4%, 3% 2%, 1%, 0.9%, 0.8%, 0.7%, 0.6%, 0.5%, 0.4%, 0.3%, 0.2%, 0.1% or 0.01% of the recited value or range of values while still describing the particular composition or solid state form.

“Substantially identical” as used herein refers to measured physical characteristics that are comparable in value or data traces that are comparable in peak position and amplitude or intensity within the scope of variations that are typically associated with sample positioning or handling or the identity of the instrument employed to acquire the traces or physical characteristics or due to other variations or fluctuations normally encountered within or between laboratory environments or analytical instrumentation.

“Substantially pure” as used herein refers to a solid state form of a compound described herein that contains less than about 3% or less than about 2% by weight total impurities, or more preferably less than about 1% by weight water, and/or less than about 0.5% by weight impurities such as decomposition or synthesis by-products or residual organic solvent.

“Essentially pure” as used herein refers to a form of a compound described herein wherein the sum of impurities or related substance in the form is less than 1%, preferably less than 0.75%, more preferably less than 0.5% and that the residual solvents and water are less than 1%, preferably less than 0.75%, more preferably less than 0.5% and still more preferably less than 0.25% by weight.

The term “crystalline forms” and related terms herein refers to the various crystalline states of a given substance, including, but not limited to, polymorphs, solvates, hydrates, mixed solvates, co-crystals and other molecular complexes. A crystalline form may also be, but is not necessarily, a mixture of various crystalline states of a given substance such as a combination of pseudopolymorph or polymorph forms, a combination of one or more polymorph forms with one or more pseudopolymorph or a combination of such forms with amorphous or non-solid state forms of the substance. Typical combinations are of two or more polymorph or pseudo polymorph forms, such a mixture of a polymorph form with a pseudopolymorph form or a mixture of a polymorph or pseudopolymorph form with amorphous material. Typically crystalline forms are typically distinguishable from each other by their XRPD patterns. Solid state forms having different crystal morphologies but essentially identical XRPD patterns are considered to be different crystalline forms, since different morphologies can exhibit different properties related to physical shape. Properties related to physical shape include dissolution rate, stability, hygroscopicity, mechanical properties such hardness, tensile strength, compatibility (tableting) and those related to handling, e.g., flow, filtering, blending and other physical or pharmaceutical properties as described herein for different polymorphs.

Embodiments disclosed herein are also meant to encompass pharmaceutically acceptable salts of Compound 1 being isotopically-labelled by having one or more atoms replaced by an atom having a different atomic mass or mass number (i.e., an “isotopic form” of the pharmaceutically acceptable salts of Compound 1). Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabeled compounds could be useful to help determine or measure the effectiveness of the compounds, by characterizing, for example, the site or mode of action, or binding affinity to pharmacologically important site of action. Certain isotopically-labeled pharmaceutically acceptable salts of Compound 1, for example, those incorporating a radioactive isotope, are useful in drug and/or substrate tissue distribution studies. The radioactive isotopes tritium (i.e. ³H), and carbon-14 (i.e., ¹⁴C) are particularly useful for this purpose in view of their ease of incorporation and ready means of detection.

Substitution with heavier isotopes such as deuterium (i.e., ²H) may afford certain therapeutic advantages resulting from greater metabolic stability, for example, increased in vivo half-life or reduced dosage requirements, and hence are preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled salts of Compound 1 can generally be prepared by conventional techniques known to those skilled in the art or by processes analogous to those described in the Examples as set out below using an appropriate isotopically-labeled reagent in place of the non-labeled reagent previously employed.

A “tautomer” refers to a proton shift from one atom of a molecule to another atom of the same molecule. Embodiments thus include tautomers of the disclosed compounds.

A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

The chemical naming protocol and structure diagrams used herein are a modified form of the I.U.P.A.C. nomenclature system, using the ACD/Name Version 9.07 software program and/or ChemDraw Ultra Version 11.0.1 software naming program (CambridgeSoft). For complex chemical names employed herein, a substituent group is typically named before the group to which it attaches. For example, cyclopropylethyl comprises an ethyl backbone with a cyclopropyl substituent. Except as described below, all bonds are identified in the chemical structure diagrams herein, except for all bonds on some carbon atoms, which are assumed to be bonded to sufficient hydrogen atoms to complete the valency.

### II. Formulations

The present disclosure provides formulations comprising Compound 1 or a pharmaceutically acceptable salt thereof.

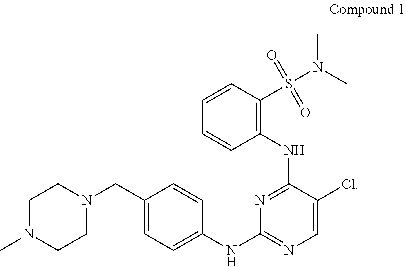

In some embodiments, the formulation is for oral administration. In some embodiments, the formulation is a solid formulation. In some embodiments, the formulation is a dry blended powder. In some embodiments, the formulation is a capsule. In some embodiments, the formulation is a capsule comprising a capsule shell and a dry blended powder enclosed within the capsule shell.

In some embodiments, the formulation comprises Compound 1 or a pharmaceutically acceptable salt thereof. In some embodiments, the Compound 1 or a pharmaceutically acceptable salt thereof is a tartrate salt.

In some embodiments, the formulation comprises from about 0.1 wt % to about 50 wt % of Compound 1 or a pharmaceutically acceptable salt thereof. In some embodiments, the formulation comprises from about 0.5 wt % to about 30 wt % of Compound 1 or a pharmaceutically acceptable salt thereof.

In some embodiments, the formulation comprises from about 0.1 wt % to about 50 wt % of a tartrate salt of Compound 1. In some embodiments, the formulation comprises from about 0.5 wt % to about 30 wt % of a tartrate salt of Compound 1. Various forms of tartrate salts of Compound 1 are described herein, for example, one form of the tartrate salt of Compound 1 is a di-tartrate salt of Compound 1; another form of the tartrate salt of Compound 1 is the sub-tartrate salt of Compound 1. In some embodiments, the di-tartrate salt of Compound 1 is the crystalline Form A di-tartrate salt of Compound 1. In some embodiments, the sub-tartrate salt of Compound 1 is the crystalline Form B sub-tartrate salt of Compound 1.

In some embodiments, the formulation comprises a unit dose of Compound 1 or a pharmaceutically acceptable salt thereof (e.g., the tartrate salt). The unit dose may comprise about 1-100 mg of Compound 1 or a pharmaceutically acceptable salt thereof. The unit dosage may comprises about 1-100 mg of the tartrate salt of Compound 1, for example about 1-100 mg of the di-tartrate salt (e.g., of crystalline Form A) of Compound 1; or for example about 1-100 mg of the sub-tartrate sale (e.g., of crystalline Form B). In some embodiments, the unit dose disclosed herein may comprise about 1 mg, 4 mg, 16 mg, 25 mg, 50 mg, 75 mg, or 100 mg of Compound 1 or a pharmaceutically acceptable salt thereof. In some embodiments, the unit dose disclosed herein may comprise about 1 mg, 4 mg, 16 mg, 25 mg, 50 mg, 75 mg, or 100 mg of the tartrate salt of Compound 1, for example about 1 mg, 4 mg, 16 mg, 25 mg, 50 mg, 75 mg, or 100 mg of the di-tartrate salt (e.g., of crystalline Form A) of Compound 1; or for example about 1 mg, 4 mg, 16 mg, 25 mg, 50 mg, 75 mg, or 100 mg of the sub-tartrate salt (e.g., of crystalline Form B) of Compound 1. In various embodiments, the amount of a tartrate salt of Compound 1 in the formulation is expressed as the weight of the salt form, i.e., inclusive of the weight of the tartrate ion(s).

In some embodiments, the formulation comprises one or more pharmaceutically acceptable excipients. In some embodiments, the formulation comprises a diluent. For example, the formulation comprises microcrystalline cellulose, lactose (e.g., lactose monohydrate), or a combination thereof. In some embodiments the formulation comprises microcrystalline cellulose and lactose monohydrate. One suitable form of microcrystalline cellulose is commercially available Avicel PH-112. Lactose monohydrate is a suitable form of lactose; however, other forms of lactose may be used in place of (or in combination with) lactose monohydrate in the various embodiments described herein.

In some embodiments, the formulation comprises from about 50 wt % to about 98 wt % of one or more diluents. In some embodiments, the formulation comprises from about 5 wt % to about 50 wt % microcrystalline cellulose. In some embodiments, the formulation comprises from about 10 wt % to about 30 wt % microcrystalline cellulose. In some embodiments, the formulation comprises from about 15 wt % to about 25 wt % of microcrystalline cellulose. In some embodiments, the formulation comprises from about 25 wt % to about 90 wt % lactose monohydrate. In some embodiments, the formulation comprises from about 50 wt % to about 80 wt % of lactose monohydrate. In some embodiments, the formulation comprises from about 50 wt % to about 75 wt % of lactose monohydrate.

In some embodiments, the formulation comprises from about 50 wt % to about 98 wt % of diluents selected from microcrystalline cellulose, lactose (e.g., lactose monohydrate), or a combination thereof. In some embodiments, the formulation comprises from about 10 wt % to about 30 wt % of microcrystalline cellulose and from about 50 wt % to about 80 wt % of lactose monohydrate. In some embodiments, the formulation comprises microcrystalline cellulose and lactose monohydrate in a ratio of from about 0.1:1 to about 1:1, e.g., from about 0.2:1 to about 0.5:1; from about 0.25:1 to about 0.35:1; or about 0.3:1. The flow properties of the diluents help to improve the overall flow of the blend during manufacture and therefore help achieve desired blend uniformity and content uniformity of the formulation.

In some embodiments, the formulation comprises a disintegrant. For example, the formulation comprises croscarmellose sodium. One suitable form of croscarmellose sodium is commercially available Ac-di-sol. In some embodiments, the formulation comprises from about 1 wt % to about 5 wt % of croscarmellose sodium. In some embodiments, the formulation comprises from about 2 wt % to about 4 wt % of croscarmellose sodium. In some embodiments, the formulation comprises about 3 wt % of croscarmellose sodium. The disintegrant reduces slug formation during capsule filling (in particular, automatic capsule filling) by breaking the slug, which ultimately leads to improved dissolution.

In some embodiments, the formulation comprises a lubricant. For example, the formulation comprises magnesium stearate. In some embodiments, the formulation comprises from about 0.5 wt % to about 2 wt % of magnesium stearate. In some embodiments, the formulation comprises about 1 wt % of magnesium stearate. The lubricant reduces sticking of the blend during further processing; for example avoids sticking to the capsule filler (e.g., automatic capsule filler) during capsule filling.

In some embodiments, the formulation comprises: Compound 1 or a pharmaceutically acceptable salt thereof, microcrystalline cellulose, lactose (e.g., lactose monohydrate), croscarmellose sodium; and magnesium stearate.

In some embodiments, the formulation comprises a tartrate salt of Compound 1. In some embodiments, the tartrate salt of Compound 1 is a di-tartrate salt of Compound 1. In some embodiments, the tartrate salt of Compound 1 is a mono-tartrate salt of Compound 1. In some embodiments, the tartrate salt of Compound 1 is a sub-tartrate salt of Compound 1.

In some embodiments, the di-tartrate salt of Compound 1 is a crystalline salt of Form A. In some embodiments, the mono-tartrate salt of Compound 1 is a crystalline salt of Form D. In some embodiments, the sub-tartrate salt of Compound 1 is a crystalline salt of Form B.

In some embodiments, the formulation comprises:

from about 0.5 wt % to about 30 wt % of Compound 1 or a pharmaceutically acceptable salt thereof;

from about 10 wt % to about 30 wt % microcrystalline cellulose;

from about 50 wt % to about 80 wt % lactose monohydrate;

from about 1 wt % to about 5 wt % croscarmellose sodium; and

from about 0.5 wt % to about 2 wt % magnesium stearate.

In some embodiments, the formulation comprises a unit dosage of crystalline Form A of a di-tartrate salt of Compound 1 of 4 mg, 25 mg, or 100 mg. In some embodiments, the formulation comprises a unit dosage of crystalline Form B of a sub-tartrate salt of Compound 1 of 1 mg, 4 mg, or 16 mg.

In some embodiments, the formulation is prepared by a direct dry blending process.

In some embodiments, the formulation is a capsule, wherein the percentages of Compound 1 or a pharmaceutically acceptable salt thereof, microcrystalline cellulose, lactose monohydrate, croscarmellose sodium, and magnesium stearate in the formulation are expressed in weight percent of the capsule exclusive of capsule shell.

In some embodiments, a capsule is provided comprising a capsule shell enclosing a dry blended powder, wherein the powder comprises tartrate salt of Compound 1, microcrystalline cellulose, lactose (e.g., lactose monohydrate), croscarmellose sodium; and magnesium stearate.

In some embodiments, the dry blended powder comprises:

from about 0.5 wt % to about 30 wt % of a di-tartrate salt of Compound 1;

from about 10 wt % to about 30 wt % microcrystalline cellulose;

from about 50 wt % to about 80 wt % lactose monohydrate;

from about 1 wt % to about 5 wt % croscarmellose sodium; and

from about 0.5 wt % to about 2 wt % magnesium stearate.

In some embodiments, provided is a formulation comprising:

crystalline Form A of a di-tartrate salt of Compound 1;

microcrystalline cellulose;

lactose monohydrate;

croscarmellose sodium; and

magnesium stearate.

In some embodiments, provided is a formulation comprising

from about 0.5 wt % to about 30 wt % of crystalline Form A of a di-tartrate salt of Compound 1;

from about 10 wt % to about 30 wt % microcrystalline cellulose;

from about 50 wt % to about 80 wt % lactose monohydrate;

from about 1 wt % to about 5 wt % croscarmellose sodium; and

from about 0.5 wt % to about 2 wt % magnesium stearate.

In some embodiments, provided is a unit dosage form comprising:

about 4 mg of crystalline Form A of a di-tartrate salt of Compound 1;

from about 30 to about 40 mg of microcrystalline cellulose;

from about 100 to about 150 mg of lactose monohydrate;

from about 4 to about 6 mg of croscarmellose sodium; and

from about 1 to about 2.5 mg of magnesium stearate.

In some embodiments, provided is a unit dosage form comprising:

about 25 mg of crystalline Form A of a di-tartrate salt of Compound 1;

from about 27 to about 37 mg of microcrystalline cellulose;

from about 80 to about 120 mg of lactose monohydrate;

from about 4 to about 6 mg of croscarmellose sodium; and

from about 1 to about 2.5 mg of magnesium stearate.

In some embodiments, provided is a unit dosage form comprising:

about 100 mg of crystalline Form A of a di-tartrate salt of Compound 1;

from about 50 to about 70 mg of microcrystalline cellulose;

from about 150 to about 250 mg of lactose monohydrate;

from about 8 to about 14 mg of croscarmellose sodium; and

from about 2 to about 5 mg of magnesium stearate.

In some embodiments, the dry blended powder comprises:

from about 0.5 wt % to about 30 wt % of a sub-tartrate salt of Compound 1;

from about 10 wt % to about 30 wt % microcrystalline cellulose;

from about 50 wt % to about 80 wt % lactose monohydrate;

from about 1 wt % to about 5 wt % croscarmellose sodium; and

from about 0.5 wt % to about 2 wt % magnesium stearate.

In some embodiments, provided is a formulation comprising:

crystalline Form B of a sub-tartrate salt of Compound 1;

microcrystalline cellulose;

lactose monohydrate;

croscarmellose sodium; and

magnesium stearate.

In some embodiments, provided is a formulation comprising from about 0.5 wt % to about 30 wt % of crystalline Form B of a sub-tartrate salt of Compound 1;

from about 10 wt % to about 30 wt % microcrystalline cellulose;

from about 50 wt % to about 80 wt % lactose monohydrate;

from about 1 wt % to about 5 wt % croscarmellose sodium; and

from about 0.5 wt % to about 2 wt % magnesium stearate.

In some embodiments, provided is a unit dosage form comprising:

about 1 mg of crystalline Form B of a sub-tartrate salt of Compound 1;

from about 30 to about 40 mg of microcrystalline cellulose;

from about 100 to about 150 mg of lactose monohydrate;

from about 4 to about 6 mg of croscarmellose sodium; and

from about 1 to about 2.5 mg of magnesium stearate.

In some embodiments, provided is a unit dosage form comprising:

about 4 mg of crystalline Form B of a sub-tartrate salt of Compound 1;

from about 30 to about 40 mg of microcrystalline cellulose;

from about 100 to about 150 mg of lactose monohydrate;

from about 4 to about 6 mg of croscarmellose sodium; and

from about 1 to about 2.5 mg of magnesium stearate.

In some embodiments, provided is a unit dosage form comprising:

about 16 mg of crystalline Form B of a sub-tartrate salt of Compound 1;

from about 30 to about 40 mg of microcrystalline cellulose;

from about 100 to about 150 mg of lactose monohydrate;

from about 4 to about 6 mg of croscarmellose sodium; and

from about 1 to about 2.5 mg of magnesium stearate.

### III. Manufacturing Processes

In some embodiments, provided herein is a process for preparing a formulation (e.g., a capsule) comprising Compound 1 or a pharmaceutically acceptable salt thereof.

In some embodiments, the process is a direct dry blend process.

In some embodiments, the process comprises directly blending Compound 1 or a pharmaceutically acceptable salt thereof with one or more excipients, e.g., one or more diluents, disintegrants, and/or lubricants. The direct blending may be done in a blender, e.g., a turbula blender. The blending may be at a rate of about 40-45 (e.g., about 42) rotations per minute (rpm). The direct blending may be sequential. For example, the Compound 1 or salt thereof may be first blended with a first diluent (e.g., lactose), then blended with a second diluent (e.g., microcrystalline cellulose), then blended with a disintegrant (e.g., croscarmellose sodium), and last blended with a lubricant (e.g., magnesium stearate).

In some embodiments, the process comprises sifting Compound 1 or a pharmaceutically acceptable salt thereof (e.g., tartrate salt) and/or sifting one or more of the excipients. For example, in some embodiments, Compound 1 or a pharmaceutically acceptable salt thereof is sifted through a 100 #sifter prior to blending. In some embodiments, the diluents (lactose, MCC), disintegrant (croscarmellose sodium) and/or lubricant (magnesium stearate) are sifted through a sifter (e.g., a 30 #, 40 #, or 60 #sifter) prior to respective addition to the blend. In some embodiments, the blended mixture is sifted again (e.g., through a 40 #sifter) after blending.

In some embodiments, the process comprises a trituration step. In some embodiments, the Compound 1 or pharmaceutically acceptable salt thereof is triturated prior to being added to the blender. The triturating may be performed in a mortar, using a pestle. In some embodiments, the Compound 1 or pharmaceutically acceptable salt thereof is triturated together with lactose (e.g., lactose monohydrate). In some embodiments, the lactose is added gradually to the API in the mortar during trituration.

In some embodiments, provided is a process for preparing a capsule formulation, comprising:

sifting Compound 1 or a pharmaceutically acceptable salt thereof;

sifting lactose monohydrate;

blending the Compound 1 or a pharmaceutically acceptable salt thereof and lactose monohydrate with microcrystalline cellulose to form a first dry blend;

blending the first dry blend with croscarmellose sodium to form a second dry blend;

blending magnesium stearate with the second dry blend to form a final dry blend; and

filling a capsule shell with the final dry blend to form the capsule formulation.

In some embodiments, the lactose monohydrate is blended gradually with the microcrystalline cellulose and Compound 1 or a pharmaceutically acceptable salt thereof.

In some embodiments, blending to form the first dry blend and/or second dry blend comprises blending at from about 40 to about 45 rpm.

In some embodiments, provided is a process for preparing a capsule formulation, comprising:

triturating Compound 1 or a pharmaceutically acceptable salt thereof and lactose monohydrate in a mortar to form a triturate;

blending the triturate with microcrystalline cellulose in a blender to form a first dry blend;

blending the first dry blend with croscarmellose sodium to form a second dry blend;

blending magnesium stearate with the second dry blend to form a final dry blend; and

filling a capsule shell with the final dry blend to form the capsule formulation.

In some embodiments, the lactose monohydrate is added gradually during trituration.

In some embodiments, the process further comprises sifting Compound 1 or a pharmaceutically acceptable salt thereof and/or lactose monohydrate prior to trituration.

In some embodiments, the process further comprises sifting the microcrystalline cellulose, croscarmellose sodium, and/or magnesium stearate prior to blending.

In some embodiments, blending to form the first dry blend and/or the second dry blend comprises blending at from about 40 to about 45 rpm.

### IV. Tartrate Salts of Compound 1

In some embodiments, the Compound 1 or a pharmaceutically acceptable salt thereof is a tartrate salt of Compound 1. For example, a formulation described herein may comprise a tartrate salt of Compound 1. As another example, a process described herein may include use of a tartrate salt of Compound 1.

In some embodiments, the tartrate salt disclosed herein may have a molar ratio of tartaric acid to Compound 1 of about 1:1 to about 2:1. For example, the tartrate salt may have a molar ratio of tartaric acid to Compound 1 of about 2:1 (di-tartrate), or alternatively about 1.2:1 (sub-tartrate) or about 1:1 (mono-tartrate). Any of the tartrate salts disclosed herein may be a salt of L-(+)-tartaric acid.

The present disclosure also provides a crystalline form of any of the tartrate salts disclosed herein. In some embodiments, the crystalline form is crystalline Form A, having a molar ratio of tartaric acid to Compound 1 of about 2:1. In some examples, Form A can be in substantially pure form. In some embodiments, the crystalline form comprises Form A. In some embodiments, the crystalline form consists essentially of Form A. In some embodiments, the crystalline form is Form B, having a molar ratio of tartaric acid to Compound 1 of about 1.2:1. In some embodiments, the crystalline form is Form D, having a molar ratio of tartaric acid to Compound 1 of about 1:1.

Any of the crystalline forms disclosed herein may have an initial purity of at least 99% and a subsequent purity of at least 99% after being stored for up to about 15 days at about 25° C.±2° C. at a relative humidity of 60±5%. In some embodiments, the crystalline form may an initial purity of at least 99% and a subsequent purity of at least 99% after being stored for up to about 15 days at about 40° C.±2° C. at a relative humidity of 75±5%.

Also within the scope of the present disclosure is a composition comprising any of the tartrate salts disclosed herein or any of the crystalline forms also disclosed herein. In some embodiments, the composition comprises Form A in substantially pure form. In some embodiments, the composition comprises at least 90% Form A by weight. In some embodiments, the composition consists essentially of crystalline Form A.

In certain embodiments, the tartrate salt is a salt of L-(+)-tartaric acid. In certain embodiments, the tartrate salt of Compound 1 is a crystalline or partially crystalline solid.

Other salt forms of Compound 1 are provided. The salt may be a pharmaceutically acceptable salt. The pharmaceutically acceptable salt of Compound 1 can be represented by the following structure:

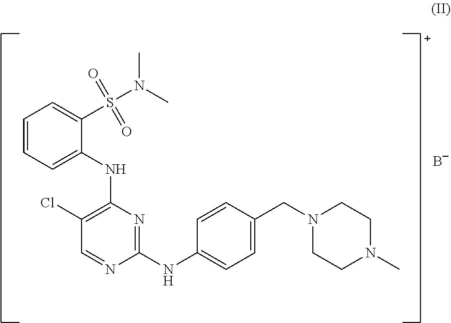

wherein B− is the conjugate base of the acid used for salt formation. In certain embodiments, the pharmaceutically acceptable salt is a phosphoric acid salt. In certain embodiments, the pharmaceutically acceptable salt is a malate salt. In certain embodiments, the pharmaceutically acceptable salt is a succinate salt. In certain embodiments, the pharmaceutically acceptable salt is a benzenesulfonate salt.

Several salts of Compound 1 were screened. The tartrate salt exhibits favorable pharmacokinetic properties, such as bioavailability. See Examples.

1. Stoichiometric and Crystalline Forms

In some embodiments, the molar ratio of tartaric acid to Compound 1 ranges from about 4:1 to about 1:4, from about 3.5:1 to about 1:3.5, from about 3.2:1 to about 1:3.2, from about 3:1 to about 1:3, from about 2.7:1 to about 1:2.7, from about 2.5:1 to about 1:2.5, from about 2.2:1 to about 1:2.2, from about 2:1 to about 1:2.2, from about 1.8:1 to about 1:2.2, from about 1.5:1 to about 1:2.2, from about 1.2:1 to about 1:2.2, from about 1.1:1 to about 1:2.2, from about 0.8:1 to about 1:2.2, from about 0.5:1 to about 1:2.2, from about 0.2:1 to about 1:2.2, from about 0.1:1 to about 1:2.2, or from about 2:1 to about 1:2.5.

In certain embodiments, the molar ratio of tartaric acid to Compound 1 is about 1:1; e.g., from about 0.8:1 to about 1.2:1. In certain embodiments, the molar ratio is 0.8:1, 0.9:1, 1:1, 1.1:1, or 1.2:1. In one embodiment, the molar ratio is 1:1. In another embodiment, the molar ratio is 1.2:1. One embodiment provides a tartrate salt having the following structure

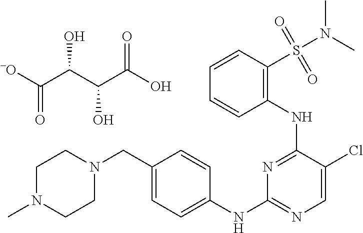

In certain embodiments, the tartrate salt of Compound 1 has one of the following structures (IIb), (IIc), (IId), (IIe), (IIf) or (IIg):

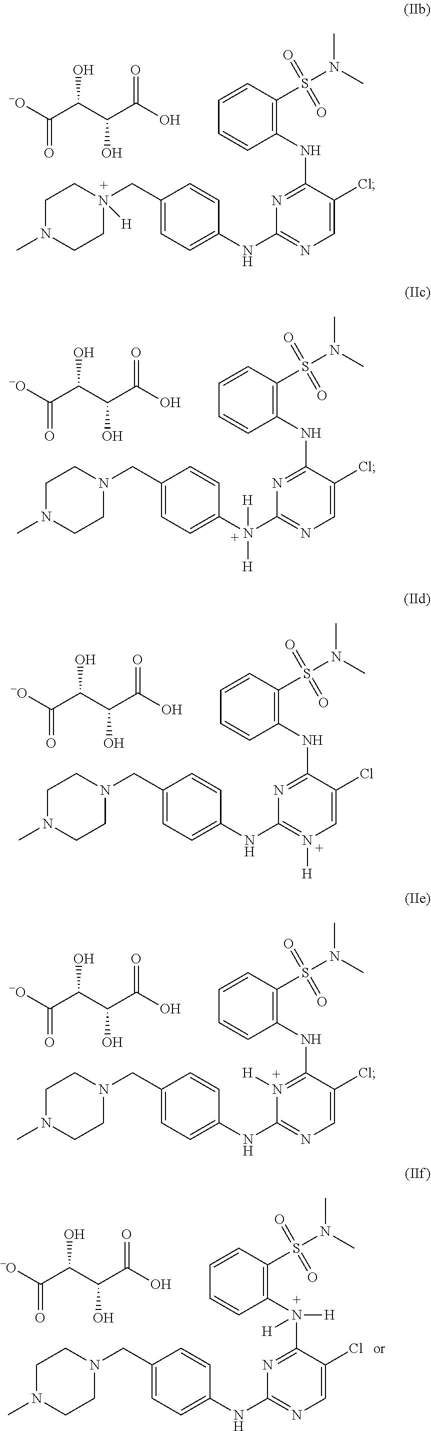

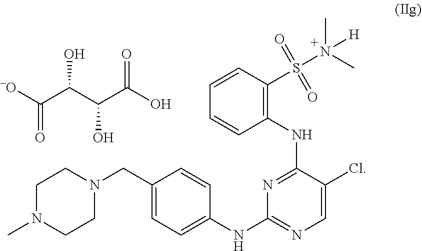

a. Form B

In certain embodiments, a tartrate salt having a stoichiometry of about 1:1 is of crystalline Form B, and is characterized by one or more of X-ray powder diffraction (XRPD), Differential Scanning Calorimetry (DSC), and Thermogravimetric Analysis (TGA). In one embodiment, Form B has a molar ratio of tartaric acid to Compound 1 of 1:1. In another embodiment, Form B has a molar ratio of tartaric acid to Compound 1 of 1.2:1. The tartrate salt of Compound 1 having a ratio of tartaric acid to Compound 1 of 1.2:1 is referred to herein as the “sub-tartrate of Compound 1.”

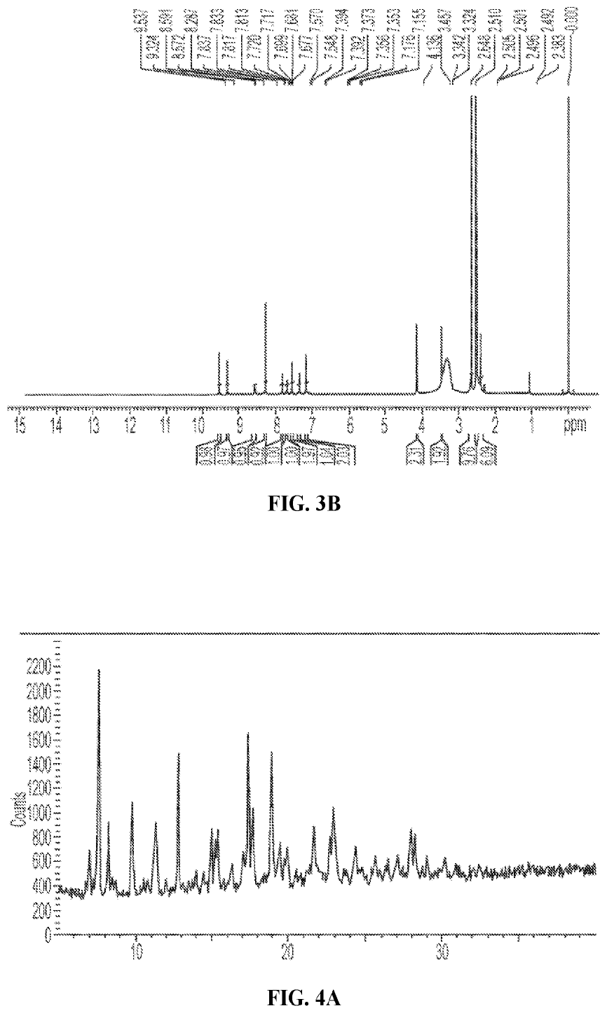

In certain embodiments, Form B is characterized by an XRPD pattern comprising two or more peaks, in units of 2-theta, selected from 7.5±0.2, 10.3±0.2, 18.9±0.2, and 19.0±0.2 at a temperature of about 22° C. In certain embodiments, the XRPD pattern of Form B comprises 2, 3, or 4 peaks selected from 7.5±0.2, 10.3±0.2, 18.9±0.2, and 19.0±0.2. In certain embodiments, the XRPD pattern is substantially identical to that of FIG. 3A. In certain embodiments, the XRPD pattern comprises one or more (e.g., 1, 2, 3, 4, or 5) additional peaks selected from the peaks listed in FIG. 7.

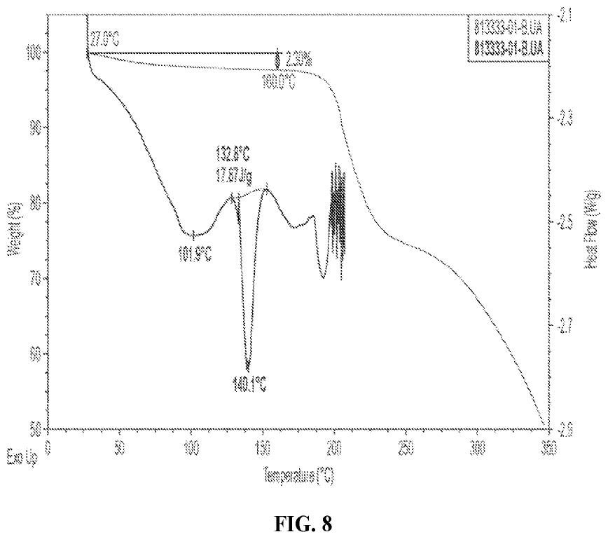

In certain embodiments, Form B is characterized by a DSC thermogram comprising an endotherm peak in units ° C. at about 101.9. In certain embodiments, the DSC thermogram comprises an endotherm peak in units ° C. at about 140.1. In certain embodiments, the DSC thermogram comprises endotherm peaks in units ° C. at about 101.9 and 140.1. In one embodiment, the DSC thermogram is substantially identical to that of FIG. 8.

In certain embodiments, Form B is characterized by a TGA thermogram showing weight loss of about 2.3% at 160° C. In one embodiment, the TGA thermogram is substantially identical to the thermogram shown in FIG. 8.

Physical and chemical properties of Form B are described in the Examples.

b. Form D

In certain embodiments, a tartrate salt having a stoichiometry of about 1:1 is of crystalline Form D, and is characterized by one or more of XRPD, DSC, and TGA. In one embodiment, Form D has a molar ratio of tartaric acid to Compound 1 of 1:1.

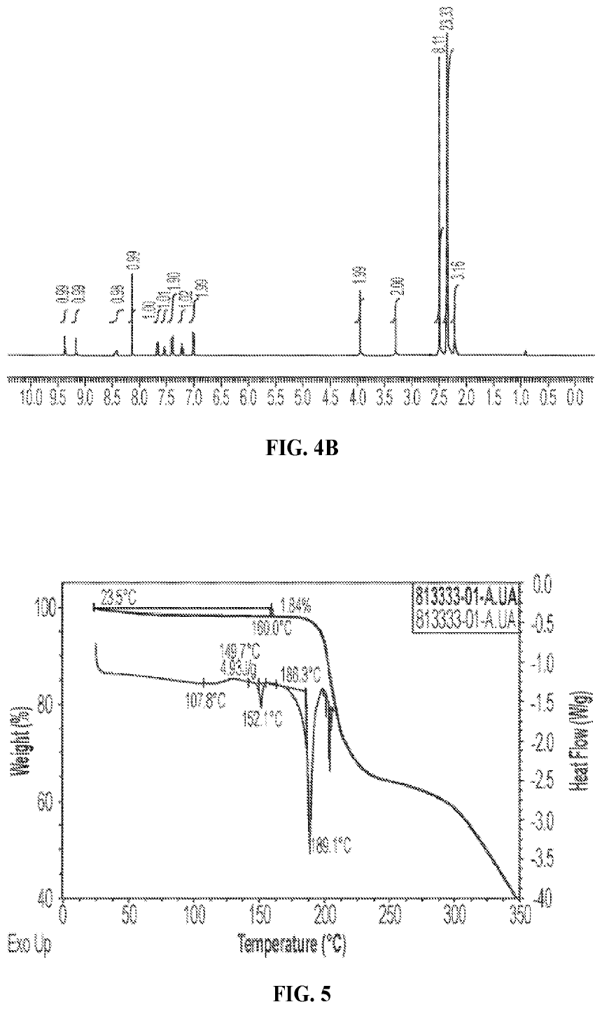

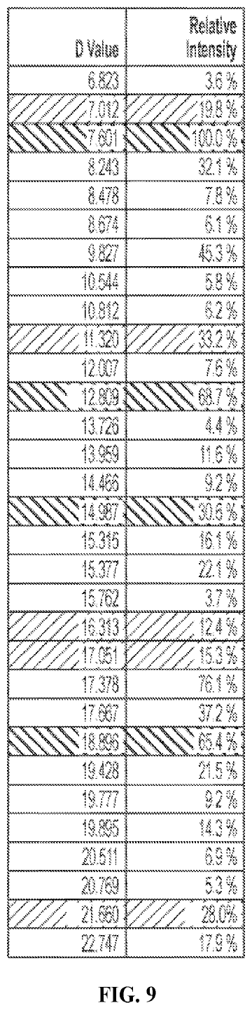

In certain embodiments, Form D is characterized by an XRPD pattern comprising peaks, in units of 2-theta, at 12.8±0.2 and 18.9±0.2 at a temperature of about 22° C. In certain embodiments, the XRPD pattern is substantially identical to that of FIG. 4A. In certain embodiments, the XRPD pattern comprises one or more (e.g., 1, 2, 3, 4, or 5) additional peaks selected from the peaks listed in FIG. 9.

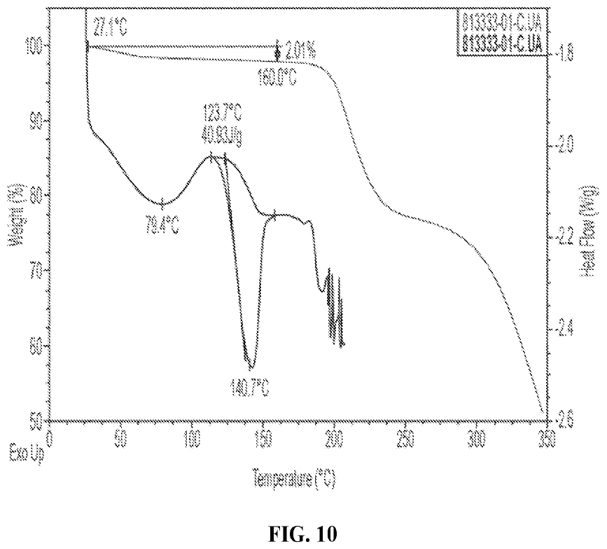

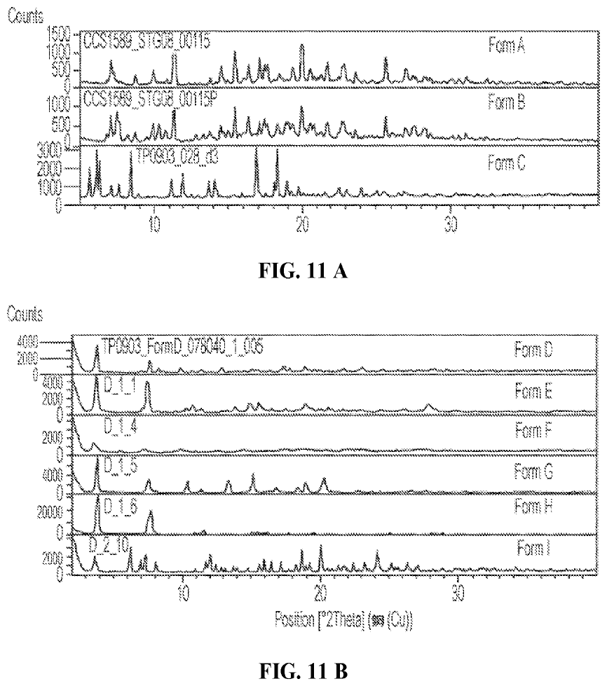

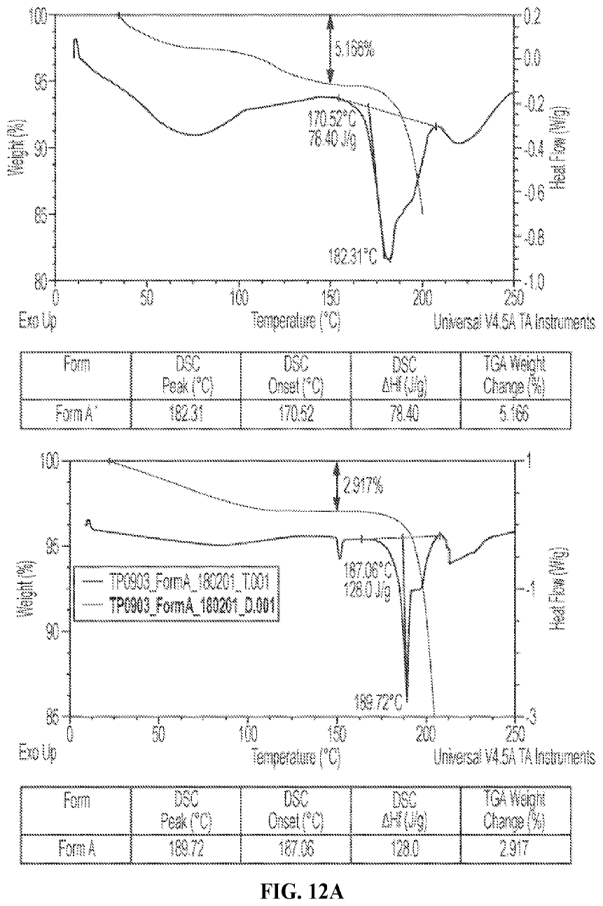

In certain embodiments, Form D is characterized an endotherm peak in units ° C. at about 79.4. In certain embodiments, the DSC thermogram comprises an endotherm peak in units ° C. at about 140.7. In certain embodiments, the DSC thermogram comprises endotherm peaks in units ° C. at about 79.4 and 140.7. In one embodiment, the DSC thermogram is substantially identical to that of FIG. 10.

In certain embodiments, Form D is characterized by a TGA thermogram showing weight loss of about 2.0% at 160° C. In one embodiment, the TGA thermogram is substantially identical to that of FIG. 10.

In certain embodiments, a tartrate salt having a stoichiometry of about 1:1 comprises Form D. In certain embodiments, a tartrate salt having a stoichiometry of about 1:1 consists essentially of Form D. In certain embodiments, Form D is essentially pure.

Physical and chemical properties of Form B are described in the Examples. Toxicokinetic and toxicology profiles of Form B are also described in the Examples.

c. Form A′

In certain embodiments, a tartrate salt having a stoichiometry of about 1.5:1 is of crystalline Form A′, and is characterized by one or more of XRPD, DSC, and TGA. In certain embodiments, the molar ratio of tartaric acid to Compound 1 is about 1.5:1; e.g., from about 1.4:1 to about 1.6:1. In certain embodiments, the molar ratio is 1.4:1, 1.5:1, or 1.6:1. In one embodiment, the molar ratio is 1.5:1. In certain embodiments, crystalline Form A′ is characterized by a DSC thermogram comprising an endotherm peak at about 182.3° C. In certain embodiments, the endotherm peak has an onset temperature of about 170.5° C.

In certain embodiments, a tartrate salt having a stoichiometry of about 1.5:1 comprises Form A′. In certain embodiments, a tartrate salt having a stoichiometry of about 1.5:1 consists essentially of Form A′. In certain embodiments, Form A′ is essentially pure.

d. Form A

In certain embodiments, a tartrate salt having a stoichiometry of about 2:1 is of crystalline Form A, and is characterized by one or more of XRPD, DSC, and TGA. In certain embodiments, the molar ratio of tartaric acid to Compound 1 ranges from about 2.2:1 to about 1.9:1. In one embodiment, the molar ratio is 2:1.

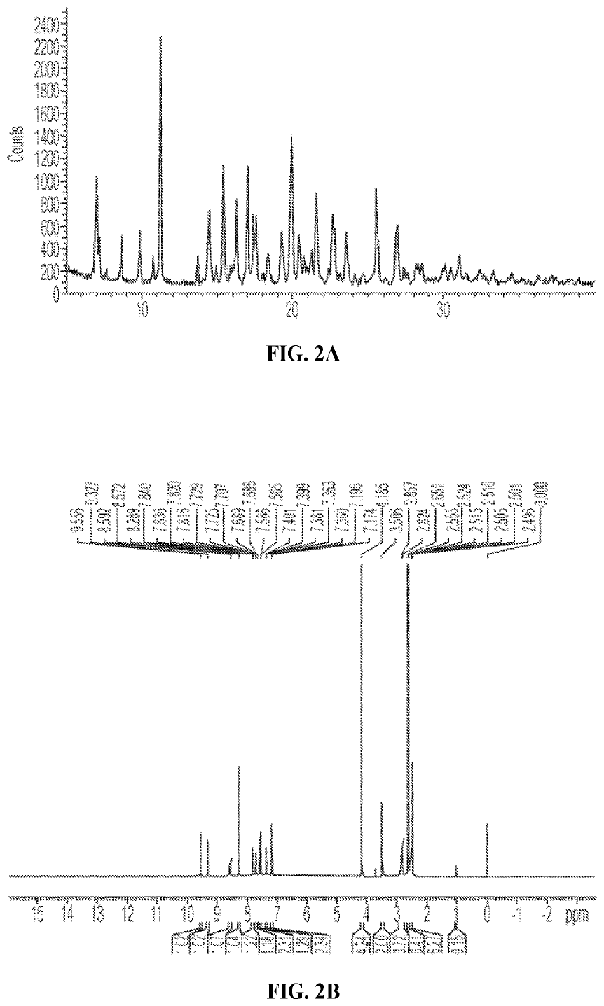

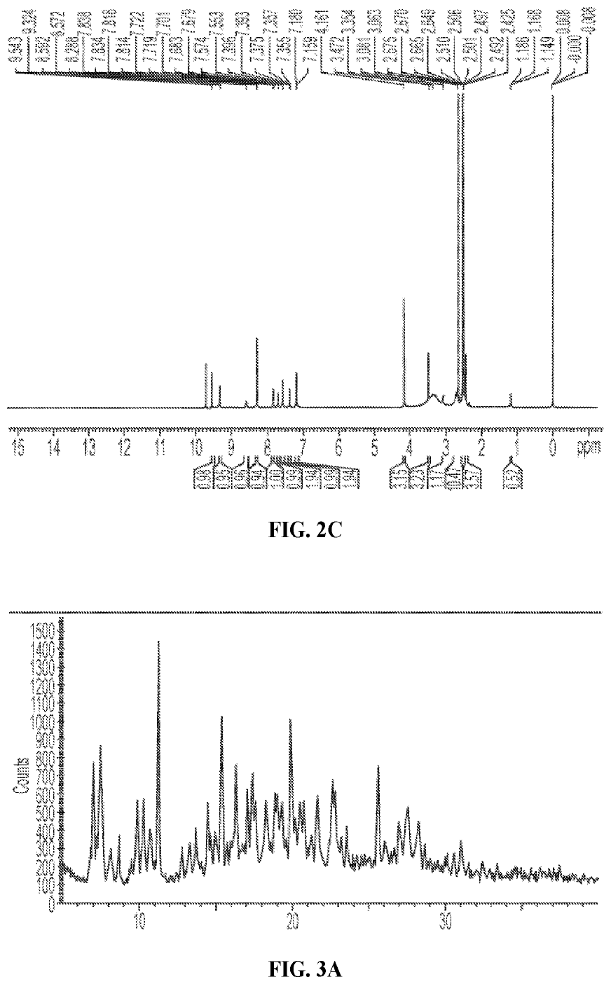

In certain embodiments, Form A is characterized by an XRPD pattern comprising three or more peaks, in units of 2-theta, selected from 7.0±0.2, 11.2±0.2, 15.4±0.2, 16.3 0.2, 17.1±0.2, 19.9±0.2, 21.6±0.2, and 25.5±0.2 at a temperature of about 22° C. In certain embodiments, the XRPD pattern of Form A comprises 3, 4, 5, 6, 7, or 8 peaks selected from 7.0±0.2, 11.2±0.2, 15.4±0.2, 16.3±0.2, 17.1±0.2, 19.9±0.2, 21.6±0.2, and 25.5±0.2. In certain embodiments, the XRPD pattern is substantially identical to that of FIG. 2A. In certain embodiments, the XRPD pattern comprises one or more (e.g., 1, 2, 3, 4, or 5) additional peaks selected from the peaks listed in Table 1.

TABLE 1
Tabulated X-ray Powder Diffractogram data from Form A

| | | Net | Gross | Relative |
|---|---|---|---|---|
| Angle | D Value | Intensity | Intensity | Intensity |
| 7.016 | 12.58927 | 872 | 1003 | 39.7% |
| 7.137 | 12.37531 | 283 | 413 | 12.9% |
| 7.671 | 11.51594 | 89.1 | 210 | 4.1% |
| 8.655 | 10.20899 | 379 | 489 | 17.3% |
| 9.869 | 8.95559 | 442 | 542 | 20.1% |
| 10.751 | 8.22221 | 215 | 312 | 9.8% |
| 11.241 | 7.86498 | 2195 | 2290 | 100.0% |
| 13.701 | 6.45790 | 223 | 312 | 10.2% |
| 14.070 | 6.28943 | 34.2 | 128 | 1.6% |
| 14.457 | 6.12183 | 595 | 692 | 27.1% |
| 14.914 | 5.93526 | 129 | 229 | 5.9% |
| 15.402 | 5.74823 | 990 | 1093 | 45.1% |
| 15.921 | 5.56201 | 114 | 222 | 5.2% |
| 16.286 | 5.43814 | 666 | 776 | 30.4% |
| 17.046 | 5.19745 | 1012 | 1124 | 46.1% |
| 17.392 | 5.09476 | 473 | 585 | 21.5% |
| 17.562 | 5.04590 | 505 | 616 | 23.0% |
| 18.035 | 4.91450 | 65.4 | 173 | 3.0% |
| 18.371 | 4.82545 | 226 | 330 | 10.3% |
| 19.280 | 4.60009 | 427 | 532 | 19.5% |
| 19.910 | 4.45589 | 1229 | 1339 | 56.0% |
| 20.445 | 4.34050 | 406 | 517 | 18.5% |
| 20.752 | 4.27683 | 172 | 282 | 7.8% |
| 20.915 | 4.24395 | 130 | 239 | 5.9% |
| 21.235 | 4.18071 | 279 | 387 | 12.7% |
| 21.579 | 4.11474 | 799 | 904 | 36.4% |
| 21.909 | 4.05363 | 38.7 | 139 | 1.8% |
| 22.383 | 3.96882 | 110 | 208 | 5.0% |
| 22.665 | 3.91998 | 512 | 609 | 23.3% |
| 23.147 | 3.83948 | 78.3 | 174 | 3.6% |
| 23.538 | 3.77658 | 417 | 509 | 19.0% |
| 24.111 | 3.68807 | 64.8 | 149 | 3.0% |
| 24.670 | 3.60583 | 95.8 | 180 | 4.4% |
| 25.533 | 3.48590 | 789 | 880 | 36.0% |
| 26.170 | 3.40239 | 44.5 | 137 | 2.0% |
| 26.904 | 3.31131 | 518 | 614 | 23.6% |
| 27.366 | 3.25638 | 114 | 213 | 5.2% |
| 27.575 | 3.23223 | 78.8 | 180 | 3.6% |
| 28.167 | 3.16561 | 163 | 266 | 7.4% |
| 28.282 | 3.15301 | 146 | 250 | 6.7% |
| 28.542 | 3.12484 | 146 | 250 | 6.7% |
| 28.960 | 3.08070 | 32.3 | 137 | 1.5% |
| 29.228 | 3.05304 | 33.5 | 138 | 1.5% |
| 29.485 | 3.02701 | 28.9 | 135 | 1.3% |
| 30.066 | 2.96986 | 155 | 265 | 7.0% |
| 30.461 | 2.93226 | 104 | 215 | 4.8% |
| 31.002 | 2.88223 | 189 | 299 | 8.6% |
| 31.440 | 2.84310 | 62.4 | 169 | 2.8% |
| 32.056 | 2.78984 | 34.6 | 139 | 1.6% |
| 32.362 | 2.76416 | 90.1 | 195 | 4.1% |
| 33.279 | 2.69008 | 99.6 | 202 | 4.5% |
| 34.511 | 2.59683 | 59.1 | 157 | 2.7% |
| 36.261 | 2.47542 | 46.4 | 136 | 2.1% |
| 37.084 | 2.42233 | 45.3 | 136 | 2.1% |
| 37.386 | 2.40348 | 33.8 | 123 | 1.5% |
| 38.983 | 2.30860 | 46.3 | 126 | 2.1% |

In certain embodiments, Form A is characterized by a DSC thermogram comprising an endotherm peak value at about 185.0° C.-194.0° C. In some embodiments, the endotherm peak value is at a temperature ranging from about 186.0° C.-193.0° C., from about 187.0° C.-192.0° C., or from about 188.0° C.-191.0° C. In some embodiments, the endotherm peak value is at about 189.1° C.

In certain embodiments, Form A is characterized by a DSC thermogram comprising an endotherm peak value at about 148.0° C.-155.0° C. In some embodiments, the endotherm peak value is at a temperature ranging from about 150.0° C.-154.0° C., from about 151.0° C.-153.0° C., or from about 151.5° C.-152.5° C. as determined by differential scanning calorimetry. In some embodiments, the endotherm peak value is at about 152.1° C.

In certain embodiments, Form A is characterized by a DSC thermogram comprising endotherm peak values at about 185.0° C.-194.0° C. and at about 148.0° C.-155.0° C. In some embodiments, the endotherm peak values are at a temperature ranging from about 186.0° C.-193.0° C., from about 187.0° C.-192.0° C., or from about 188.0° C.-191.0° C. and from about 150.0° C.-154.0° C., from about 151.0° C.-153.0° C., or from about 151.5° C.-152.5° C. In some embodiments, the endotherm peak values are at about 189.1° C. and at about 152.1° C.

In certain embodiments, Form A is characterized by a DSC thermogram comprising an endotherm peak in units ° C. at about 107.8. In certain embodiments, the DSC thermogram comprises an endotherm peak in units ° C. at about 152.1. In certain embodiments, the DSC thermogram comprises an endotherm peak in units ° C. at about 189.1. In certain embodiments, the DSC thermogram comprises endotherm peaks in units ° C. at about 107.8, about 152.1, and about 189.1. In certain embodiments, the DSC thermogram is substantially identical to that of FIG. 5.

In certain embodiments, Form A is characterized by a TGA thermogram showing weight loss of about 1.8% at 160° C. In certain embodiments, the TGA thermogram is substantially identical to that of FIG. 5.

In certain embodiments, a tartrate salt having a stoichiometry of about 2:1 comprises Form A. In certain embodiments, a tartrate salt having a stoichiometry of about 2:1 consists essentially of Form A. In certain embodiments, Form A is essentially pure. Physical and chemical properties of Form A are described in the Examples.

e. Forms C, E, F, G, H, and I

The tartrate salt of Compound 1 exists in other crystalline forms, as summarized in Table 2.

TABLE 2
Crystalline forms of tartrate salts of Compound 1

| | Solvent | Slurry | Slurry | | | DSC | Moisture |
|---|---|---|---|---|---|---|---|
| Crystal | screen from | screen from | screen from | | Stability | Onset | sorption |
| form | Form A | Form A | Form D | Stoichiometry | by HPLC | (° C.) | isotherm |
| A | +++ | +++ | Not formed | 1:1.5 | Stable | 170.52 | hygroscopic |
| | | | | 1:2 | Stable | 187.06 | hygroscopic |
| B | + | + | | 1:1.2 | | 131.62 | hygroscopic |
| C | + | Not formed | | | | 165.76 | hygroscopic |
| D | Not formed | | ++ | 1:1 | Stable | 127.43 | hygroscopic |
| E | | | + | | | 102.33 | |
| F | | | + + | | | 61.06 | |
| G | | | + | | | 128.61 | |
| H | | | + | | | 139.36 | |
| I | | | + | | | 118.60 | |

+++: Formed in three solvent systems or more
++: Formed in a few solvent systems
+: Formed in one solvent systems or only at the higher temperature

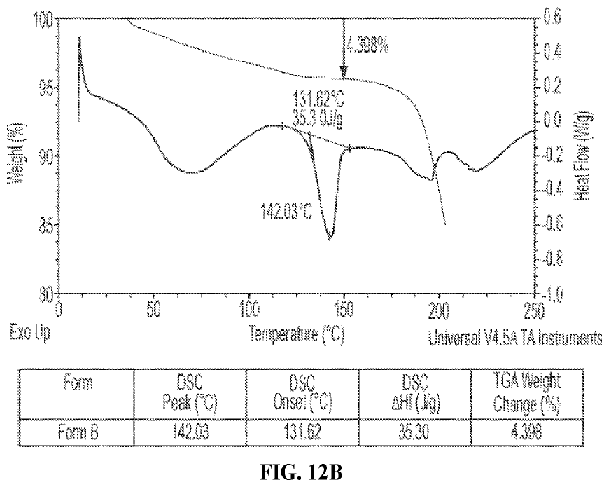

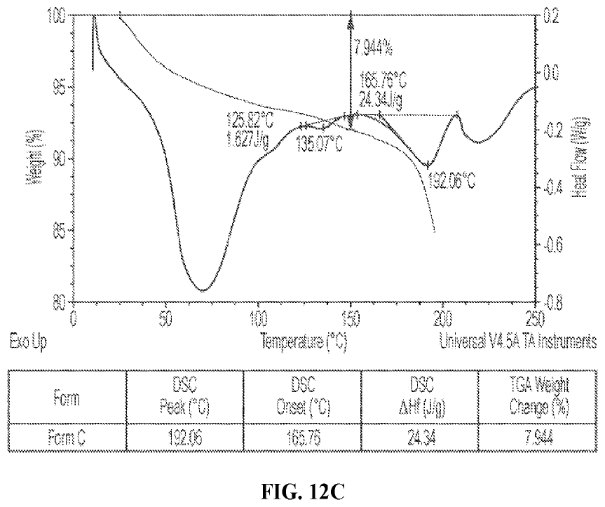

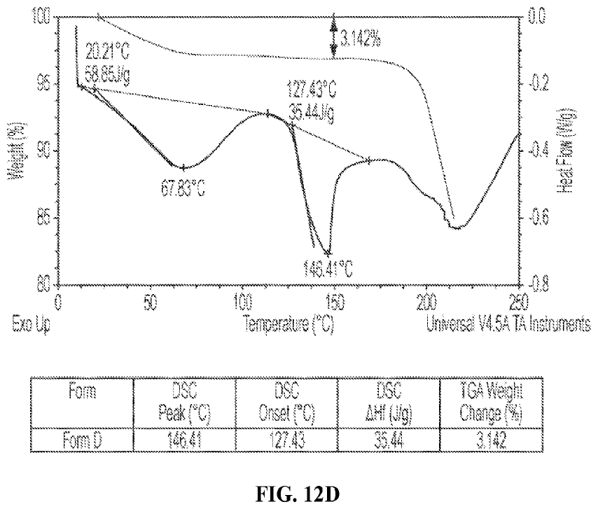

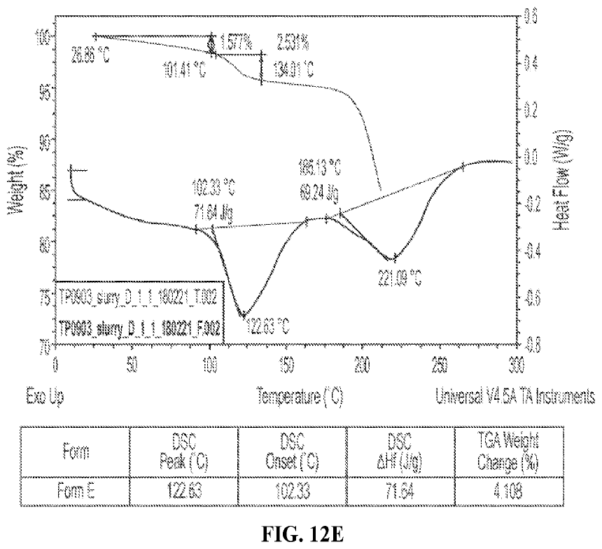

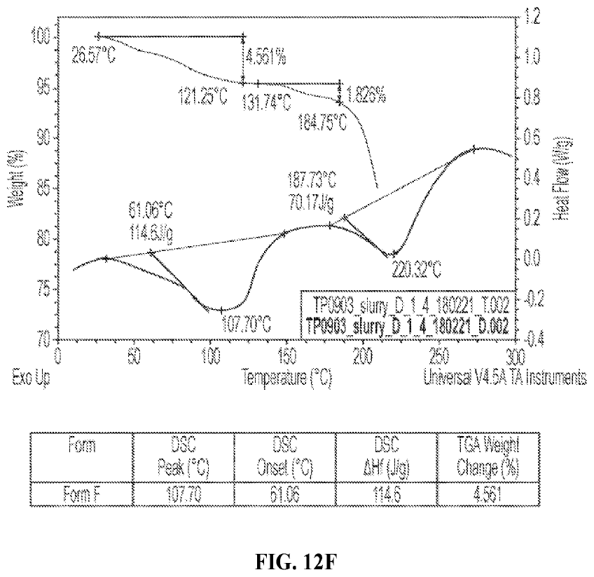

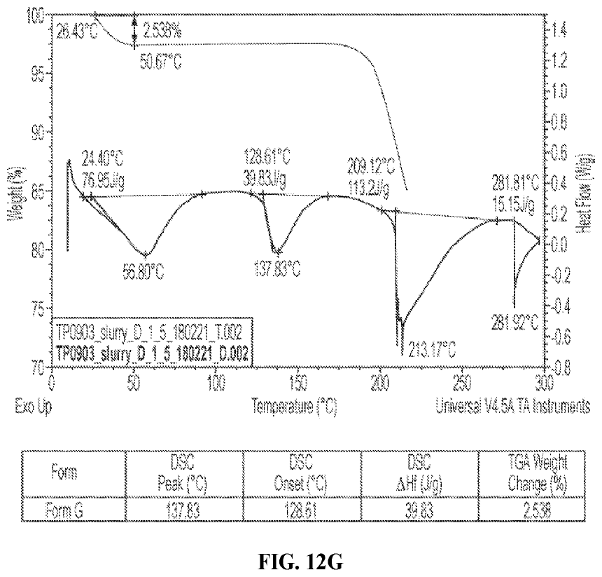

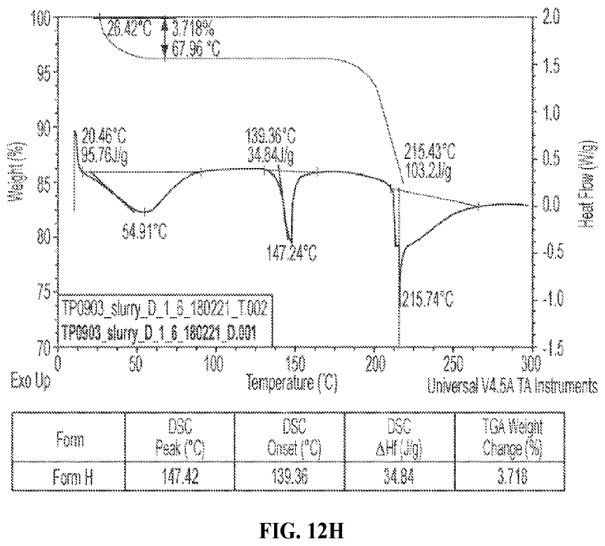

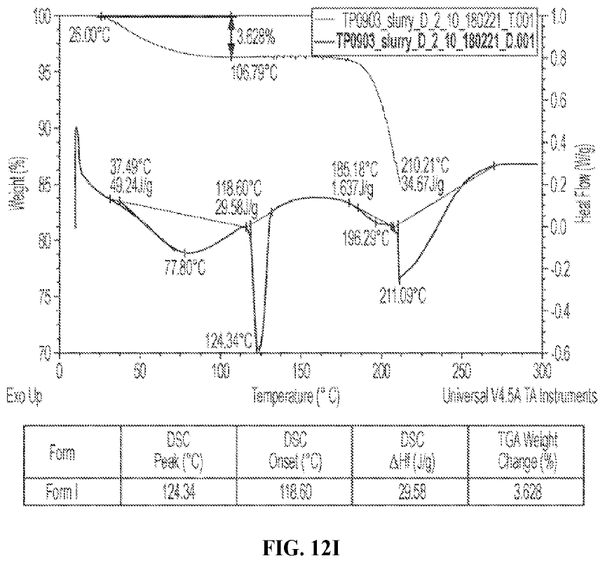

From polymorph screening studies, ten crystal forms, Form A (1:2), A′(1:1.5), B, C, D, E, F, G, H and I, were assigned by XRPD patterns as shown in FIG. 11. The thermal behavior (DSC/TGA charts) for these crystalline forms are shown in FIGS. 12A-12I.

The Slurry Screens and Solvent Screens described below were performed according to the methods of Example 11.

TABLE 3
Slurry Screening of Form A

| Room Temperature | | | 50° C. | | |
|---|---|---|---|---|---|
| | | Crystal | | | Crystal |
| Run | Solvent | Form | Run | Solvent | Form |
| 1-1 | Methanol | A | 2-1 | Methanol | A |
| 1-2 | Ethanol | A | 2-2 | Ethanol | A |
| 1-3 | 2-Propanol | A | 2-3 | 2-Propanol | A |
| 1-4 | 2-Propanol-H₂O | A | 2-4 | 2-Propanol-H₂O | A |
| | (5:1) | | | (5:1) | |
| 1-5 | H₂O | B | 2-5 | H₂O | — |
| 1-6 | Methanol-H₂O | A | 2-6 | Methanol-H₂O | — |
| | (5:1) | | | (5:1) | |
| 1-7 | Ethanol-H₂O | A | 2-7 | Ethanol-H₂O | A |
| | (5:1) | | | (5:1) | |
| 1-8 | Methanol-H₂O | A | 2-8 | Methanol-H₂O | A |
| | (10:1) | | | (10:1) | |
| 1-9 | Acetonitrile-H₂O | — | 2-9 | Acetonitrile-H₂O | — |
| | (1:1) | | | (1:1) | |
| 1-10 | Acetonitrile-H₂O | A | 2-10 | Acetonitrile-H₂O | A |
| | (10:1) | | | (10:1) | |

TABLE 4
Solvent Screen of Form A′

| Run | Crystal Form | Solvent |
|---|---|---|
| 001 | B | H₂O |
| 002 | — | Methanol |
| 003 | A | Ethanol |
| 004 | A | 2-Propanol |
| 005 | A | 2-Butanol |
| 006 | A | Chloroform |
| 007 | A | Acetonitrile |
| 008 | A | 1,2-Dimethoxy |
| | | ethane |
| 009 | A | Tetrahydrofuran |
| 010 | A | Butyl methyl |
| | | ether |
| 011 | A | Cyclopentyl |
| | | methyl ether |
| 012 | A | Ethyl acetate |
| 013 | A | Propyl acetate |
| 014 | A | Acetone |
| 015 | A | Heptane |
| 016 | A | Chlorobenzene |
| 017 | A | Toluene |
| 018 | A + C | H₂O-Methanol |
| | | (1:1) |
| 019 | — | H₂O-Ethanol |
| | | (1:1) |
| 020 | C | H₂O-2-Propanol |
| | | (1:1) |
| 021 | — | H₂O-Acetonitrile |
| | | (1:1) |
| 022 | — | H₂O-Acetone |
| | | (1:1) |
| 023 | Amorphous | H₂O-Methanol |
| | | (1:10) |
| 024 | Amorphous | H₂O-Ethanol |
| | | (1:10) |
| 025 | Amorphous + A | H₂O-2-Propanol |
| | | (1:10) |
| 026 | Amorphous + A | H₂O-Acetonitrile |
| | | (1:10) |
| 027 | Amorphous + A | H₂O-Acetone |
| | | (1: 10) |
| 028 | C | H₂O-2-Propanol |
| | | (1:5) |
| 029 | — | H₂O-Acetonitrile |
| | | (1:5) |
| 030 | — | H₂O-Acetone |
| | | (1:5) |

TABLE 5
Slurry Screen of Form A′

| Room Temperature | | | 50° C. | | |
|---|---|---|---|---|---|
| | | Crystal | | | Crystal |
| Run | Solvent | Form | Run | Solvent | Form |
| 1-1 | Methanol | A | 2-1 | Methanol | A |
| 1-2 | Ethanol | A | 2-2 | Ethanol | A |
| 1-3 | 2-Propanol | A | 2-3 | 2-Propanol | A |
| 1-4 | Acetone | A | 2-4 | Acetone | A |
| 1-5 | Acetonitrile | A | 2-5 | Acetonitrile | A |
| 1-6 | 2-Propanol-H₂O | A | 2-6 | 2-Propanol-H₂O | A |
| | (10:1) | | | (10:1) | |
| 1-7 | 2-Propanol-H₂O | A | 2-7 | 2-Propanol-H₂O | A |
| | (5:1) | | | (5:1) | |
| 1-8 | Methyl tert-butyl | A | 2-8 | Methyl tert-butyl | A |
| | ether | | | ether | |
| 1-9 | 2-Propanol-H₂O | A | 2-9 | 2-Propanol-H₂O | — |
| | (1:1) | | | (1:1) | |

TABLE 6
Slurry Screen of Form D

| Room Temperature | | | 50° C. | | |
|---|---|---|---|---|---|
| | | Crystal | | | Crystal |
| Run | Solvent | Form | Run | Solvent | Form |
| 1-1 | Methanol | E | 2-1 | Methanol | F |
| 1-2 | Ethanol | D | 2-2 | Ethanol | D |
| 1-3 | 2-Propanol | D | 2-3 | 2-Propanol | D |
| 1-4 | 2-Propanol-H₂O(5:1) | F | 2-4 | 2-Propanol-H₂O(5:1) | F |
| 1-5 | H₂O | G | 2-5 | H₂O | G |
| 1-6 | Methanol-H₂O(5:1) | H | 2-6 | Methanol-H₂O(5:1) | — |
| 1-7 | Ethanol-H₂O(5:1) | F | 2-7 | Ethanol-H₂O(5:1) | F |
| 1-8 | Ethanol-H₂O(10:1) | D + F | 2-8 | Ethanol-H₂O(10:1) | F |
| 1-9 | Acetonitrile-H₂O | — | 2-9 | Acetonitrile-H₂O | — |
| | (1:1) | | | (1:1) | |
| 1-10 | Acetonitrile-H₂O | F | 2-10 | Acetonitrile-H₂O | I |
| | (10:1) | | | (10:1) | |

In the solvent screen shown in Table 4 (above), Form C was formed by the recrystallization of Form A′ with the mixture of H₂O and alcohol, such as methanol and 2-propanol. About 10% weight loss and a broad endotherm peak was observed in the thermal analysis chart as shown in FIG. 12C. That suggested that Form C might be a solvate with alcohol and the alcohol was eliminated depending on the increase of temperature.

Form E was formed in the slurry screen from Form D only in methanol at room temperature, as shown in Table 6. About 4% weight loss was observed in the thermal analysis chart as shown in FIG. 12E.

Form F was formed in the slurry screen from Form D in the mixture of alcohol and H₂O at room temperature and 50° C. as shown in Table 6. About 6%-weight loss was observed in the thermal analysis chart as shown in FIG. 12F.

Form G was formed in the slurry screen from Form D in H₂O at room temperature and 50° C. as shown in Table 6. The thermal analysis chart was provided in FIG. 12G.

Form H was formed in the slurry screen from Form D only in Methanol-H₂O (5:1) at room temperature, as shown in Table 6. The thermal analysis chart is provided in FIG. 12H.

Form I was formed in the slurry screen from Form D only in acetonitrile-H₂O (10:1) at 50° C., as shown in Table 6. Thermal analysis data is provided in FIG. 12I.

### V. Preparation of Compound 1 and Crystalline Tartrate Salts

Embodiments of Compound 1, the tartaric acid salt of Compound 1 and polymorphs thereof (e.g., Form A) can be prepared according to the General Reaction Scheme below, wherein each occurrence of X is a halide or pseudohalide (e.g., triflate, nonaflate, mesylate, tosylate, etc.). Certain intermediates useful for preparation of a tartaric acid salt of Compound 1 can be prepared according to methods described in WO 2012/135800, which is incorporated herein by reference in its entirety. As shown in the General Reaction Scheme, compounds of structure A1 can be purchased from commercial sources or prepared according to methods familiar to one of ordinary skill in the art, including those provided in the Examples (see, e.g., Example 6). Reaction of A1 with amine reagent A′ (which is prepared according to known methods or purchased from commercial sources) yields A2. Phenyl nitro compound A2 can then be converted to the aniline A3, which is coupled with A4 to yield the pyrimidine containing product A5. The pyrimidine containing A5 is then coupled to aniline compound A6 to afford A7. A7 can then be reduced as necessary (e.g., using BH₃.THF) and activated (e.g., using thionyl chloride, Comins' reagent) to yield compound A8 (i.e., Compound 1). The compound A8 may then be purified and converted to the desired polymorph/salt by adding the appropriate acid (e.g., tartaric) under the appropriate conditions (e.g., heating cooling following two re-slurry purification steps).

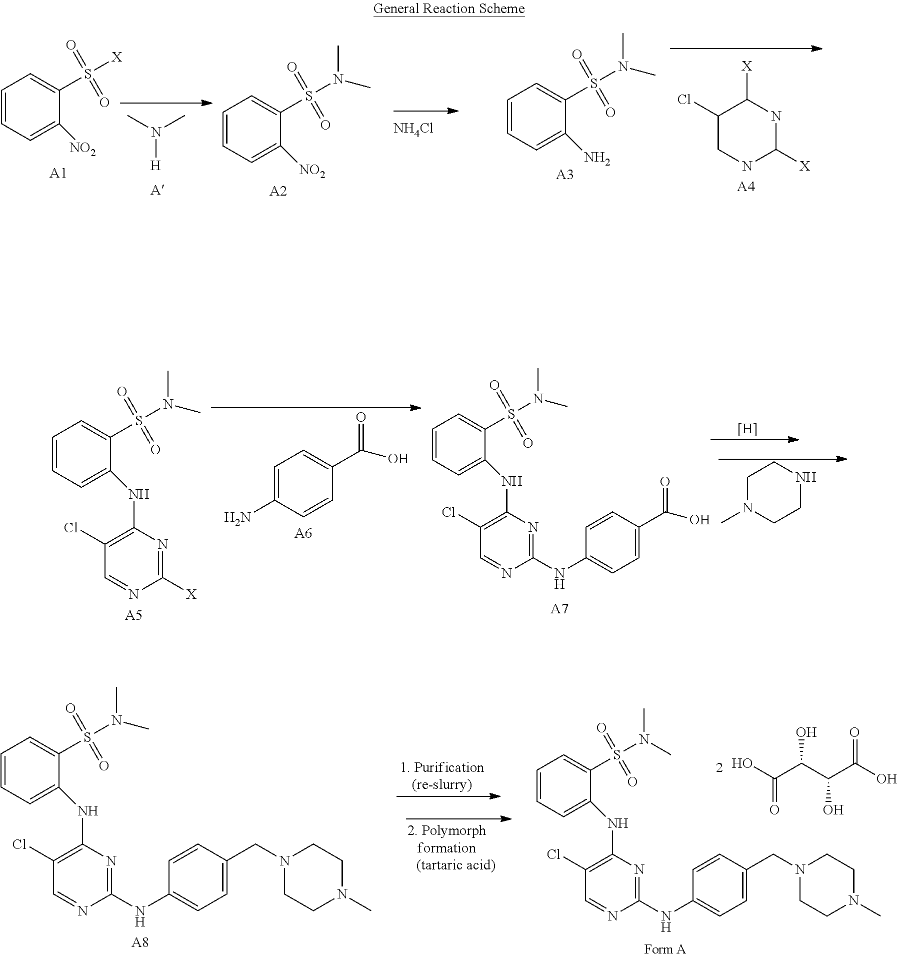

It should be noted that the General Reaction Scheme only depicts an exemplary method for preparation of a tartaric acid salt of Compound 1 and other methods are available, including methods for preparation of a tartaric acid salt of Compound 1 using different reagents, and/or different intermediates etc.

Embodiments and features of the present disclosure may be further understood from the following examples, which should not be construed as limiting the scope of the disclosure.

### EXAMPLES

### Example 1: Di-Tartrate Capsule Formulations

The di-tartrate salt of Compound 1 (Form A) was formulated into three dose strengths. Increasing amounts of drug substance were formulated into three similar blends and filled into capsules. The 25-mg and 100-mg doses were composed of slightly less microcrystalline cellulose and lactose to compensate for the larger portions of API in the dosage strengths.

Components used in the manufacturing of all three strengths of the drug product are provided in Table 7, Table 8, and Table 9 for the 4-mg, 25-mg, and 100-mg dosage strengths, respectively. The amount of lactose monohydrate was adjusted to compensate for the assay and water content of drug substance.

TABLE 7
Compound 1 Di-Tartrate Capsules 4-mg Batch

| | | Standard | | Standard | Adjusted |
|---|---|---|---|---|---|
| | | Qty. | % | Qty. | Qty. |
| No. | Ingredients | mg/Capsule | w/w | g/batch | g/batch |
| 1. | Compound 1 di-tartrate | 4.00 | 2.4 | 108.0 | 113.0 ^{a} |
| 2. | Microcrystalline | 36.80 | 21.6 | 993.6 | 993.6 |
| | Cellulose | | | | |
| 3. | Lactose, Monohydrate | 122.40 | 72.0 | 3304.8 | 3300.0 ^{a} |
| 4. | Croscarmellose | 5.10 | 3.0 | 137.7 | 137.7 |
| 5. | Magnesium Stearate | 1.700 | 1.0 | 45.9 | 45.9 |

| | | | | |
|---|---|---|---|---|
| Total | 170.0 | 100.00 | 4590.0 | 4590.0 |

| | | | |
|---|---|---|---|
| 6. | Empty HCG Swedish | 50.00 | Not applicable |
| | Orange Capsule Size 3 | | |
| | ACG | | |
| | Total Capsule Weight | 220.0 | |

^{a} API Qty. is adjusted on the basis of assay, water content and residual solvent of API, which is compensated with lactose monohydrate.

TABLE 8
Compound 1 Di-Tartrate Capsules 25-mg Batch

| | | Standard | | Standard | Adjusted |
|---|---|---|---|---|---|
| | | Qty. | % | Qty. | Qty. |
| No. | Ingredients | mg/Capsule | w/w | g/batch | g/batch |
| 1. | Compound 1 di-tartrate | 25.00 | 14.7 | 400.0 | 418.4 ^{a} |
| 2. | Microcrystalline | 32.20 | 18.9 | 515.2 | 515.2 |
| | Cellulose | | | | |
| 3. | Lactose, Monohydrate | 106.0 | 62.4 | 1696.0 | 1677.8 ^{a} |
| 4. | Croscarmellose | 5.10 | 3.0 | 81.6 | 81.6 |
| 5. | Magnesium Stearate | 1.70 | 1.0 | 27.2 | 27.2 |

| | | | | |
|---|---|---|---|---|
| Total | 170.0 | 100.0 | 2720.0 | 2720.0 |

| | | | |
|---|---|---|---|
| 6. | Empty HCG | 50.00 | NA |
| | Orange/Grey Capsule | | |
| | Size 3 ACG | | |
| | Total Capsule Weight | 220.0 | |

^{a} API Qty. is adjusted on the basis of assay, water content and residual solvent of API, which is compensated with lactose monohydrate.

TABLE 9
Compound 1 Di-Tartrate Capsules 100-mg Batch

| | | Standard | | Standard | Adjusted |
|---|---|---|---|---|---|
| | | Qty. | % | Qty. | Qty. |
| No. | Ingredients | mg/Capsule | w/w | g/batch | g/batch |
| 1. | Compound 1 di-tartrate | 100.0 ^{a} | 26.3 | 600.0 | 627.4 ^{a} |
| 2. | Microcrystalline | 61.0 | 16.1 | 366.0 | 366.0 |
| | Cellulose | | | | |
| 3. | Lactose, Monohydrate | 203.8 | 53.6 | 1222.8 | 1195.4 ^{a} |
| 4. | Croscarmellose | 11.40 | 3.0 | 68.40 | 68.40 |
| 5. | Magnesium Stearate | 3.800 | 1.0 | 22.8 | 22.80 |

| | | | | |
|---|---|---|---|---|
| Total | 170.0 | 380.0 | 100.0 | 2280.0 |

| | | | |
|---|---|---|---|
| 6. | Empty HCG Brown | 90.00 | NA |
| | Opaque Capsule Size 0 | | |
| | ACG | | |
| | Total Capsule Weight | 470.0 | |

^{a} API Qty. is adjusted on the basis of assay, water content and residual solvent of API, which is compensated with lactose monohydrate.

### Example 2: Sub-Tartrate Capsule Formulations

The sub-tartrate salt of Compound 1 (Form B) was formulated into three dose strengths—1 mg, 4 mg, and 16 mg.

Components used in the manufacturing of all three strengths of the drug product are provided in Table 10.

TABLE 10
Compound 1 sub-tartrate (Form B) capsules

| Strength | 1 mg | 4 mg | 16 mg |
|---|---|---|---|
| Drug Substance | | | |
| Compound 1 sub- | 1.000 | 4.000 | 16.00 |
| tartrate (Form B) | | | |
| Excipients | | | |
| Microcrystalline | 37.40 | 36.80 | 34.40 |
| Cellulose | | | |
| Lactose, | 124.8 | 122.4 | 112.8 |
| Monohydrate | | | |
| Croscarmellose | 5.100 | 5.100 | 5.100 |
| Sodium | | | |
| Magnesium Stearate | 1.700 | 1.700 | 1.700 |
| Subtotal | 170.0 | 170.0 | 170.0 |
| HG Capsule | 46.00 | 46.00 | 46.00 |
| Size of capsule | No. 3 | No. 3 | No. 3 |
| Color of capsule | White Opaque | Swedish Orange | Dark Green |
| Total Capsule weight | 216.0 | 216.0 | 216.0 |

### Example 3: Direct Dry Blend Manufacturing Process for Capsules

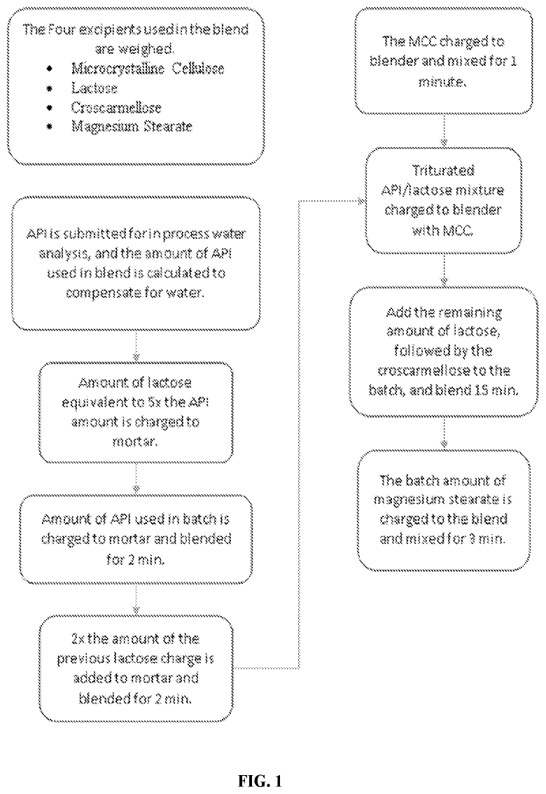

A direct dry blend manufacturing process was used to prepare the dry blended powder to be filled into capsule shells. In general terms, lactose was triturated into the Compound 1 drug substance. This mixture was charged to a blender containing microcrystalline cellulose. Croscarmellose sodium, followed by magnesium stearate were then sifted directly into the blend. Capsules were filled using an automatic capsule filling machine. A flow diagram of the process is provided in FIG. 1.

### Example 3A: The Following Steps were Followed

a. The batch quantity of microcrystalline cellulose was placed in the mixing vessel of the Turbula blender and mixed for 1 minute at 42 rpm.
b. Using the pestle, the mortar was coated with a portion (<1 g) of the lactose monohydrate.
c. Lactose monohydrate equivalent approximately ten times the weight of the drug substance was placed into the mortar.
d. Batch quantity of the drug substance was added to the stainless steel mortar
e. The drug substance and lactose monohydrate of step (d) were blended in mortar for approximately 2 minutes using the pestle.
f. Lactose monohydrate, equivalent to twice the weight of lactose monohydrate of step (e) was taken to mortar and blended for approximately 2 minutes using the pestle.
g. Lactose monohydrate, equivalent to twice the weight of Lactose Monohydrate of step (f) was taken to mortar and blended for approximately 2 minutes using the pestle.
h. Triturate from mortar was transferred to Turbula blender containing microcrystalline cellulose.
i. The mortar and pestle were further rinsed with the remaining quantity of lactose monohydrate and this mixture was transferred to the mixing vessel containing microcrystalline cellulose.
j. The batch quantity of croscarmellose sodium was sifted through a (30 #) sifter into the mixing vessel of the Turbula blender and blended at 42 rpm for 15 minutes.
k. The batch quantity of magnesium stearate was passed through 60 #sifter and added to the blend from step (j).
l. The blend from step (k) was filled into capsule shells.

### Example 3B: The Following Steps were Followed

1. Blend Preparation
   a. Sift API (Compound 1 di-tartrate) through 100 #sifter and excipient through 40 #sifter.
   b. Add microcrystalline cellulose in the mixing vessel of the turbula blender and run the blender for 5 minute at 42 rpm.
   c. Add API and lactose monohydrate (equivalent to the weight of API) quantity in turbula blender mix it for 5 minute at 42 rpm.
   d. Add same quantity of lactose monohydrate (A) (above step) and add into turbula blender mix it for 5 minute at 42 rpm.
   e. Add same quantity of lactose monohydrate (A) (above step) and add into turbula blender mix it for 5 minute at 42 rpm.
   f. Remove the blend from turbula blender and sift through 40 #sifter and again add into turbula blender.
2. Blending: Add remaining quantity of lactose monohydrate and ac-di-sol into turbula blender and mix it for 15 minute at 42 rpm
3. Lubrication: Add sifted Magnesium Stearate in Turbula blender and blend at 42 rpm for 3 minutes.
4. Capsule Filling: Automatic capsule filling machine will be used for capsule filling. Tapping will be applied as needed to achieve the target filling of capsules. If require clean the tapping pin between the capsule filling activity.
5. Dedusting and metal detection check: To ensure the target filling and quality of product, the capsules will pass through Combo Deduster and Metal Detection machine.
6. Polishing: Polish the filled capsules manually using lint free cloth. Visually check for proper polishing. Ensure that no capsule is damaged during polishing.
7. Weight sorting and elegance check: To ensure the target filling and quality of product, the capsules will be sort by weight. Use 315 capsules after weight soring of filled capsules, and observe the capsules visually for defects.

### Example 4: Salt Screening

1 mL of solvent was added to 25 μg of Compound 1 and 1 equivalent of the desired acid. The solvents used were water and N-Methyl-2-pyrrolidone (NMP). The salt screen was performed using the Crystal16 equipment. The program allows heating up to 60° C. followed by controlled cooling to 5° C. with 0.1° C. per minute. Two series of slurry experiments were initiated using water and dichloromethane as a solvent. These experiments were stirred for 3 days at 20° C. If applicable, the solvent were evaporated in a vacuum oven to isolate the solids for XRPD and further analysis as provided below.

### X-Ray Powder Diffraction (XRPD)

The X-ray powder diffraction studies were performed using a Bruker AXS D2 PHASER in Bragg-Brentano configuration, equipment #1549. Using a Cu anode at 30 kV, 10 mA; sample stage standard rotating; monochromatisation by a Kβ-filter (0.5% Ni). Slits: fixed divergence slits 1.0 mm (=0.61°), primary axial Soller slit 2.5°, secondary axial Soller slit 2.5°. Detector: Linear detector LYNXEYE with receiving slit 5° detector opening. The standard sample holder (0.1 mm cavity in (510) silicon wafer) has a minimal contribution to the background signal.

Measurement conditions: scan range 5-45° 2θ, sample rotation 5 rpm, 0.5 s/step, 0.010°/step, 3.0 mm detector slit; and all measuring conditions are logged in the instrument control file. As system suitability, corundum sample A26-B26-S(NIST standard) is measured daily.

The software used for data collection is Diffrac.Commander v3.3.35. Data analysis is done using Diffrac.Eva V3.0. No background correction or smoothing is applied to the patterns. The contribution of the Cu-Kα2 is stripped off using the Diffrac.Eva software.

### Thermo Gravitational Analysis/Differential Scanning Calorimetry (TGA/DSC)

The TGA/DSC studies were performed using a Mettler Toledo TGA/DSC1 STARe System with a 34-position auto sampler, equipment #1547.

The samples were made using aluminum crucibles (40 μl; pierced). Typically, 5-10 mg of sample was loaded into a pre-weighed aluminum crucible and was kept at 30° C. for 5 minutes, after which it was heated at 10° C./min from 30° C. to 300° C. A nitrogen purge of 40 ml/min was maintained over the sample. As system suitability check Indium and Zinc are used as references.

The software used for data collection and evaluation is STARe Software v10.00 build 2480. No corrections are applied to the thermogram.

### Differential Scanning Calorimetry (DSC)

The DSC studies were performed using a Mettler Toledo DSC1 STARe System, equipment #1564.

The samples were made using aluminum crucibles (40 μl; pierced). Typically 1-8 mg of sample was loaded onto a pre-weighed aluminum crucible and was kept at 30° C. for 5 minutes, after which it was heated at 10° C./min from 30° C. to 350° C. and kept at 350° C. again. A nitrogen purge of 40 ml/min was maintained over the sample. As system suitability check Indium and Zinc are used as references.

The software used for data collection and evaluation is STARe Software v10.00 build 2480. No corrections are applied to the thermogram.

### Microscopy

The microscopy studies were performed using an AxioVert 35M, equipped with an AxioCamERc 5s, equipment #1612. The microscope is equipped with four lenses, being Zeiss A-Plan 5×/0.12, Zeiss A-Plan 10×/0.25, LD A-Plan 20×/0.30 and Achros TIGMAT 32×/0.40. Data collection and evaluation is performed using Carl Zeiss Zen AxioVision Blue Edition Lite 2011 v1.0.0.0 software.

A small amount of sample is loaded on an object glass and spread until a thin layer is obtained.

### Dynamic Vapour Sorption (DVS)

The Dynamic Vapour Sorption studies were performed using a Surface Measurement Systems Ltd. DVS-1 No Video, equipment #2126. The sample is loaded into balance pan, typically 20-30 mg, and equilibrated at 0% RH. After the material has dried the RH is increased with 10% per step for 1 hour per increment, ending at 95% RH. After completion of the sorption cycle, the sample was dried using the same method. The software used for data collection is DVSWin v3.01 No Video. Data analysis is performed using DVS Standard Analysis Suite v6.3.0 (Standard).

From these experiments in total 16 unique XRPD patterns, or forms, were obtained. From the salt screen with water as a solvent and controlled cooling with the Crystal16, 11 unique forms were obtained including salt forms from phosphoric acid, tartaric acid (i.e., (+)-L-tartaric acid), fumaric acid, malic acid (i.e., (−)-L-malic acid), succinic acid, ethane-1,5-disulfonic acid, p-toluenesulfonic acid, methanesulfonic acid, benzenesulfonic acid, ethanesulfonic acid and benzoic acid, respectively. Additional acids were tested yielding the corresponding salt of Compound 1. These additional acids included hydrochloric acid, sulfuric acid, L-aspartic acid, maleic acid, glutamic acid, citric acid, D-glucuronic acid, glycolic acid, D-gluconic acid, L-ascorbic acid, adipic acid, naphthalene-1,5-disulfonic acid and naphthalene-2-sulfonic acid.

A salt screen with NMP as a solvent and controlled cooling with the Crystal16 did lead to one unique form from sulfuric acid, at low yield and not enough material could be obtained for further analysis.

The slurry experiments with dichloromethane as a solvent lead to 4 new forms from maleic acid, malic acid, succinic acid, and ethane-1,5-disulfonic acid, respectively. The solids from the salt screens were isolated for XRPD analysis, each unique new pattern was analyzed using DSC-TGA, microscopy, FT-IR, ¹H NMR and HPLC. In this studies, some salt forms were found to be non-reproducible and others showed reduced crystallinity after DVS.

Five forms formed from phosphoric acid, tartaric acid, malic acid, succinic acid, and benzenesulfonic acid were selected for scale up experiments, up to 500 mg, for further analysis.

Upon screening of numerous salt forms of Compound 1, tartrate salt of Compound 1 was identified as a suitable salt form of the compound for pharmaceutical uses. Surprisingly, PK studies showed improved bioavailability of the tartrate salt and the PhysChem properties measured showed the tartrate as having desirable physical properties including good stability. See Examples below.

### Example 5: Pharmacokinetic Testing of Salt Forms

The 5 salt forms of Compound 1 described in Example 4 (i.e., formed from phosphoric acid, tartaric acid, malic acid, succinic acid and benzenesulfonic acid) were tested to determine their pharmacokinetic (PK) profiles. Fasted male Sprague-Dawley rats were dosed with an oral formulation of each salt form as well as the free base form. Plasma concentration was tested at 5 minutes, 0.25, 0.5 1, 2, 4, 8, 12 and 24 hours post-dose. The data (mean values) for the 5 different salt forms and the free base is included in Table 11, below.

TABLE 11
PK data comparison for 5 representative salt forms of Compound 1.

| | | | | | | Benzene |
|---|---|---|---|---|---|---|
| | Compound | Phosphate | Tartrate | Malate | Succinate | sulfonate |
| | 1 | Salt | Salt | Salt | Salt | Salt |
| Dose | 18.2 | 14.3 | 18.7 | 18.6 | 18.7 | 17.6 |
| (PO, mg/kg) | | | | | | |
| Cₘₐₓ (ng/mL) | 116 | 103 | 174 | 134 | 79.9 | 117 |
| Tₘₐₓ (hours) | 0.833 | 1.00 | 0.667 | 0.500 | 0.833 | 0.667 |
| AUC_{0-24 hours} | 458 | 531 | 581 | 515 | 470 | 408 |
| (ng · h/mL) | | | | | | |
| Bioavailability | 26.9 | 47.2 | 42.9 | 37.3 | 32.9 | 32.4 |
| (%) | | | | | | |

As the data in Table 11 show, the tartrate salt unexpectedly had one of the best overall PK profiles, having the highest Cₘₐₓ, highest AUC for 0-24 hours, and second highest bioavailability. Because the salt obtained from phosphoric acid showed undesirable stability characteristics, it appears that the tartrate salt has the best overall profile as a drug substance.

### Example 6: Pharmacokinetic Study—Free Base v. Tartrate Salt

Fasted male Sprague-Dawley rats were dosed with the free base and the tartrate salt form of Compound 1 in 20% solutol. The free base was formulated at 5.0 mg/mL (PO) and dosed by oral gavage (18.2 mg/kg). The tartrate salt of Compound 1 was formulated at 6.5 mg/mL to account for the added weight of the tartrate component of the salt and dosed by oral gavage (14.5 mg/kg).

Plasma samples were taken at 0.25, 0.5, 1, 2, 4, 8, 12, and 24 hours post dose and analyzed for the concentration of Compound 1 by LC-MS/MS with reference to a previously determined standard curve. Pharmacokinetic parameters were calculated using a non-compartmental approach with Phoenix WinNonlin 6.3 (Pharsight, Mountain View Calif.).

It was observed that the tartrate salt was superior to the free base having a higher peak of bioavailability than the free base formulation. These data suggest that the tartrate salt of Compound 1 may be more useful in vivo than the free base form. Additionally, the tartrate salt form shows superior Cm and AUC parameters while maintaining an equivalent toxicity profile to the free base form at equal doses. That is, the tartrate salt of Compound 1 allows for higher drug plasma levels without additional toxicity. Pharmacokinetic data for the tartrate salt v. the free base is included in Table 12, below (nominal dose of 20 mg/kg).

TABLE 12
PK data comparing the free base of Compound 1 to the tartrate salt.

| | Free Base | Tartrate Salt |
|---|---|---|
| Dose (PO, ^{mg}/_{kg}) | 18.2 | 18.7 |
| Cₘₐₓ (^{ng}/_{mL}) | 116 | 174 |
| Tₘₐₓ (hours) | 0.833 | 0.667 |
| AUC_{0-24 hours} (^{ng·h}/_{mL}) | 458 | 581 |
| Bioavailability (%) | 26.9 | 42.9 |

### Example 7: Synthesis of a Tartrate Salt of Compound 1

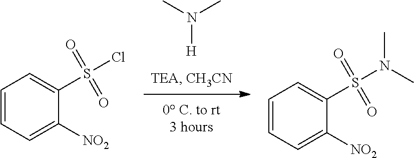

2-nitrobenzenesulfonyl chloride was combined with triethylamine and dimethylamine in acetonitrile under the reaction conditions shown to afford N,N-dimethyl-2-nitrobenzenesulfonamide in 82% yield.

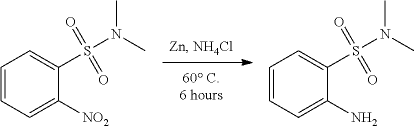

N,N-dimethyl-2-nitrobenzenesulfonamide was combined with zinc and ammonium chloride in methanol under the reaction conditions shown to afford 2-amino-N,N-dimethylbenzenesulfonamide in 99% yield.

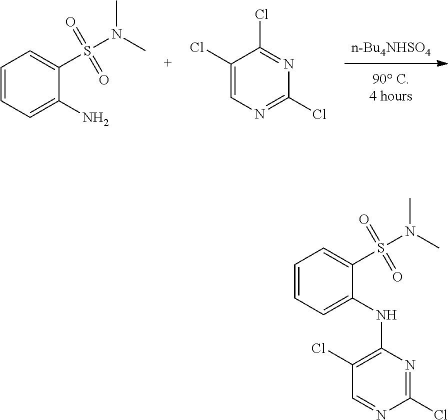 [SMILES: CN(C)S(=O)(=O)c1ccccc1N ; Clc1ncc(Cl)c(Cl)n1 ; "n-Bu4NHSO4" ; CN(C)S(=O)(=O)c1ccccc1Nc1nc(Cl)ncc1Cl]

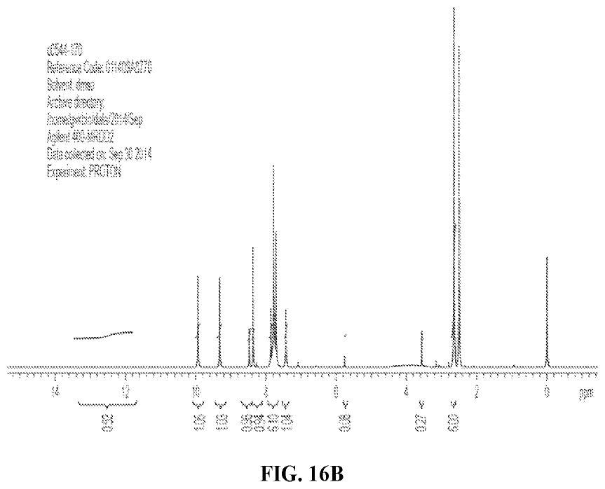

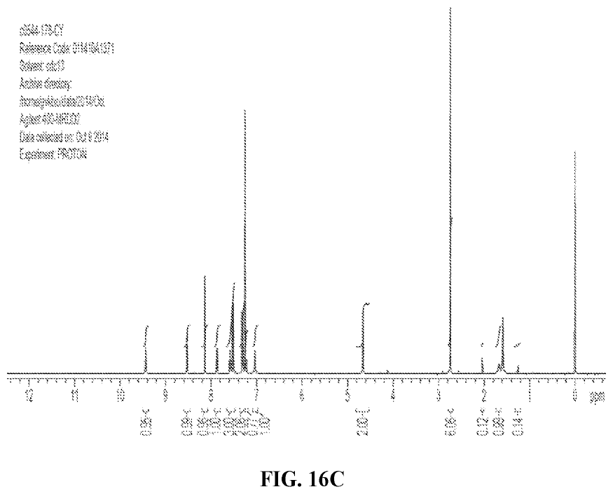

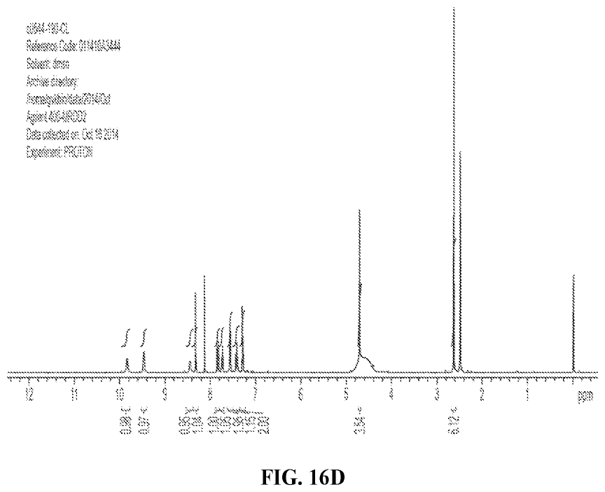

N,N-dimethylbenzenesulfonamide was combined with 2,4,5-trichloropyrimidine and tetrabutylammoniumhydrogen sulfate under the reaction conditions shown to afford 2-((2,5-dichloropyrimidin-4-yl)amino)-N,N-dimethylbenzenesulfonamide in 32% yield. ¹HNMR characterization data is shown in FIG. 16A.

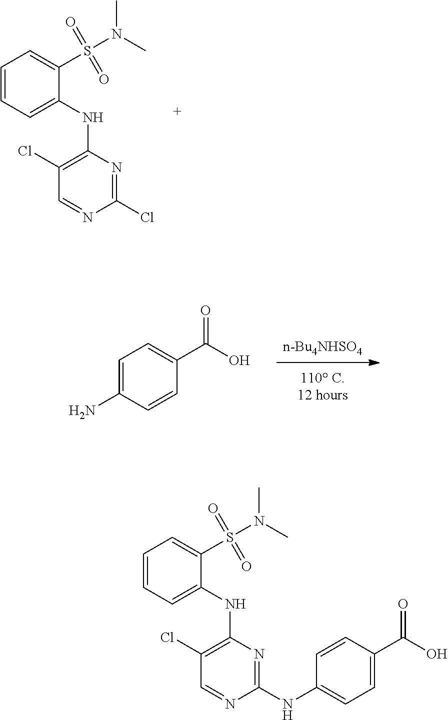 [SMILES: CN(C)S(=O)(=O)c1ccccc1Nc1nc(Cl)ncc1Cl ; Nc1ccc(C(=O)O)cc1 ; "n-Bu4NHSO4" ; CN(C)S(=O)(=O)c1ccccc1Nc1nc(Nc2ccc(C(=O)O)cc2)ncc1Cl]

2-((2,5-dichloropyrimidin-4-yl)amino)-N,N-dimethylbenzenesulfonamide was combined with 4-aminobenzoic acid and tetrabutylammoniumhydrogen sulfate under the reaction conditions shown to afford 4-((5-chloro-4-((2-(N,N-dimethylsulfamoyl)phenyl)amino)pyrimidin-2-yl)amino)benzoic acid in 85% yield. ¹HNMR characterization data is shown in FIG. 16B.

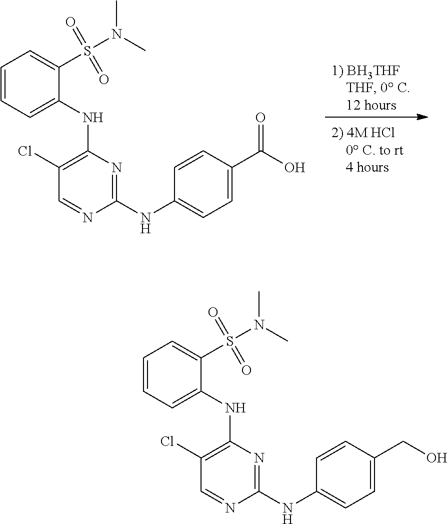

4-((5-chloro-4-((2-(N,N-dimethylsulfamoyl)phenyl)amino)pyrimidin-2-yl)amino)benzoic acid was combined with borane in tetrahydrofuran (1M) under the reaction conditions shown followed by treatment with 4 M hydrochloric acid under the reaction conditions shown to afford 2-((5-chloro-2-((4-(hydroxymethyl)phenyl)amino)pyrimidin-4-yl)amino)-N,N-dimethylbenzenesulfonamide in 80% yield. ¹HNMR characterization data is shown in FIG. 16C.

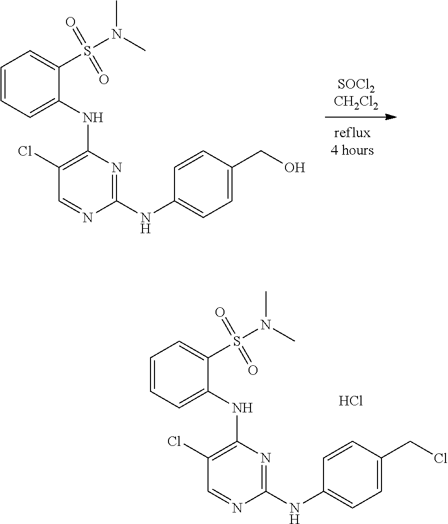 [SMILES: CN(C)S(=O)(=O)c1ccccc1Nc1nc(Nc2ccc(CO)cc2)ncc1Cl ; "SOCl2CH2Cl2" ; CN(C)S(=O)(=O)c1ccccc1Nc1nc(Nc2ccc(CCl)cc2)ncc1Cl ; Cl]

2-((5-chloro-2-((4-(hydroxymethyl)phenyl)amino)pyrimidin-4-yl)amino)-N,N-dimethylbenzenesulfonamide was combined with thionyl chloride in dichloromethane under the reaction conditions shown to afford 2-((5-chloro-2-((4-(chloromethyl)phenyl)amino)pyrimidin-4-yl)amino)-N,N-dimethylbenzenesulfonamide in 90% yield. ¹HNMR characterization data is shown in FIG. 16D.

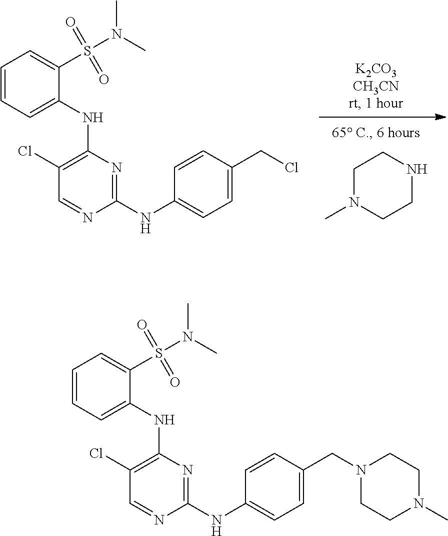

2-((5-chloro-2-((4-(chloromethyl)phenyl)amino)pyrimidin-4-yl)amino)-N,N-dimethylbenzenesulfonamide was combined with potassium carbonate and 1-methylpiperazine in acetonitrile under the reaction conditions shown to afford 2-((5-chloro-2-((4-((4-methylpiperazin-1-yl)methyl)phenyl)amino)pyrimidin-4-yl)amino)-N,N-dimethylbenzenesulfonamide (i.e., Compound 1) in 80% yield.

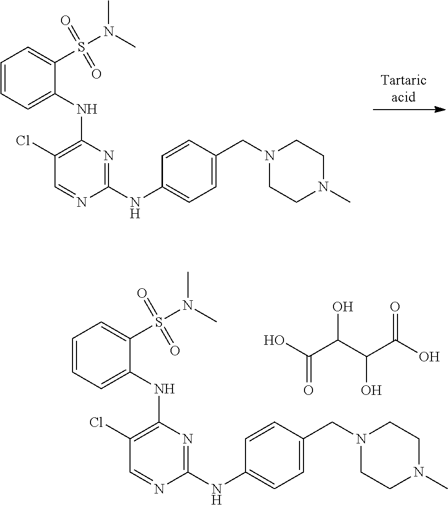

2-((5-chloro-2-((4-((4-methylpiperazin-1-yl)methyl)phenyl)amino)pyrimidin-4-yl)amino)-N,N-dimethylbenzenesulfonamide (i.e., Compound 1) was treated with tartaric acid to afford 2-((5-chloro-2-((4-((4-methylpiperazin-1-yl)methyl)phenyl)amino)pyrimidin-4-yl)amino)-N,N-dimethylbenzenesulfonamide mono-tartrate salt.

### Example 8: Polymorphs of Tartrate Salts of Compound 1

The polymorphs of the present disclosure can be prepared in view of the novel methods, reaction schemes and examples provided herein (see, e.g., Example 9), together with synthetic methods known in the art of synthetic organic chemistry, or by variations thereon as appreciated by those skilled in the art. The reactions are performed in a solvent or solvent mixture appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound or polymorph of the disclosure

The starting materials are generally available from commercial sources such as Sigma Aldrich or other commercial vendors, or are prepared as described in this disclosure, or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), Larock, R. C., Comprehensive Organic Transformations, 2^{nd}-ed., Wiley-VCH Weinheim, Germany (1999), or Beilsteins Handbuch der organischen Chemie, 4, Aufl. ed. Springer-Verlag, Berlin, including supplements (also available via the Beilstein online database)).

In the preparation of the polymorphs of Compound 1, protection of remote functionality of intermediates may be necessary. The need for such protection will vary depending on the nature of the remote functionality and the conditions of the preparation methods. The need for such protection is readily determined by one skilled in the art. For a general description of protecting groups and their use, see Greene, T. W. et al., Protecting Groups in Organic Synthesis, 4th Ed., Wiley (2007).

Additionally, the polymorphs of present disclosure exhibit valuable pharmacological properties, which can be demonstrated at least by using any one of the following test procedures. Accordingly, polymorphs of the present disclosure were assessed in biochemical assays as set forth below. Data was acquired according to the parameters listed below:

### X-Ray Powder Diffraction (XRPD):

PANalytical XRPD instrument. The solid sample was spread on a zero-background Si sample holder. The XRPD parameters used are listed below in Table 13:

TABLE 13
Settings for acquiring XRPD data

| Parameter | Value for Data Acquisition |
|---|---|
| X-Ray Wavelength | Cu, kα |
| | Ka1 (Å): 1.540598 |
| | Ka2: (Å): 1.544426 |
| | Kα2/Kα1 intensity ratio: 0.50 |
| X-ray tube setting | 45kV, 40 mA |
| Divergence slit | ⅛° |
| Scan mode | continuous |
| Scan range (°2θ) | 3°-40° |
| Step Size (°2θ) | 0.263 |
| Scan step time (s) | 50 |
| Test time(s) | ~5 minutes 4 seconds |

### TGA and DSC:

TGA data were collected using a TA 5500 TGA from TA Instruments and DSC was performed using a TA 2500 DSC from TA Instruments. Detailed parameters used are listed in Table 14 below.

TABLE 14
Instrument settings for acquiring TGA and DSC data

| Parameters | TGA | DSC |
|---|---|---|
| Method | Ramp | Ramp |
| Sample pan | Aluminum, open | Aluminum, crimped |
| Temperature | RT - desired temperature | 25° C. - desired temperature |
| Heating rate | 10° C./min | 10° C./min |
| Purge gas | N₂ | N₂ |

### HPLC:

Agilent 1260 HPLC was utilized and detailed chromatographic conditions for purity and solubility measurement are listed in Table 15 below.

TABLE 15
HPLC settings for acquiring data

| Parameters | Value | |
|---|---|---|
| HPLC | Agilent 1260 with DAD detector | |
| Column | Ascentis Express C18, 4.6 mm × 100 mm, 2.7 μm | |
| Mobile phase | A: 0.1% H₃PO₄ in H₂O | |
| | B: Acetonitrile | |
| Gradient table | Time (min) | % B |
| | 0.0 | 10 |
| | 6.0 | 95 |
| | 8.0 | 95 |
| | 8.1 | 10 |
| | 10.0 | 10 |

| Run time | 10.0 | min |
|---|---|---|
| Post time | 0.0 | min |
| Flow rate | 1.5 | mL/min |
| Injection volume | 10 | μL |
| Detector wavelength | UV at 210 nm | |
| Column temperature | 40° | C. |

| | |
|---|---|
| Sampler temperature | ambient |
| Diluent | acetonitrile:H₂O (1:1, v/v) |

### Example 9: Preparation of Crystalline Form A

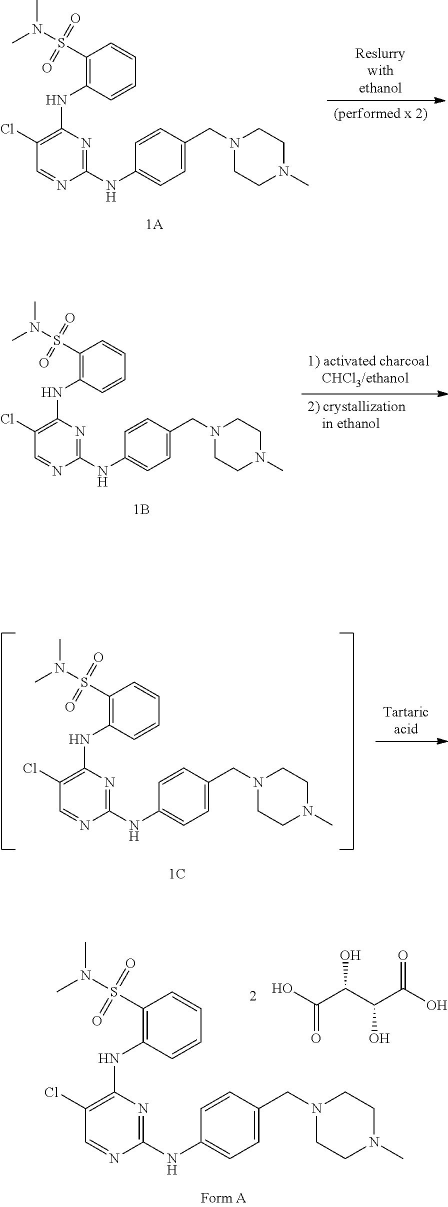

Compound 1A was obtained according to methods known in the art and met purity specifications, however, ¹H-NMR analysis showed that the material included about 30% of triethylamine, which was used in in a previous synthetic step. Applicant discovered that re-slurry in hot ethanol efficiently removed triethylamine. The re-slurry step is performed as follows:

A mixture of 1A and ethanol was heated at 70° C. for 2 hours and then slowly cooled to 20° C. over 5 hours. The slurry was filtered and dried under vacuum to provide purified 1B. This isolated crystal was subjected to a reprocessing procedure to provide 1 C.

The reprocessing procedure included dissolving 1 C in a chloroform and ethanol mixture and activated charcoal was added. The resulting slurry was stirred at room temperature for 1 hour and filtered. The filtered solid was washed, combined with filtrate and the solvents were removed by distillation. Then ethanol was added, and distillation repeated to remove chloroform. After the distillation, resulting slurry was cooled and filtered to give purified 1C. The obtained material was dissolved with mixture of anisole and ethanol at 70° C. Ethanol solution of tartaric acid was then added to this solution and subsequently seeded with Form A. The resulting slurry is cooled to 20° C. and filtered, washed with ethanol, dried to afford the polymorph of a tartaric acid salt of Compound 1. The purity of the desired product was assessed to be 99.5% by HPLC.

¹H NMR (400 MHz, DMSO-d6) δ=9.56 (s, 1H), 9.33 (s, 1H), 8.58 (s, J=8.0 Hz, 1H), 8.29 (s, 1H), 7.83 (dd, J=8.0, 1.6 Hz, 1H), 7.71 (td, J=7.2, 1.4 Hz), 7.57 (d, J=8.4 Hz, 2H), 7.38 (td, J=7.2 Hz, J=1.0 Hz), 7.18 (d, J=8.4 Hz, 2H), 4.19 (s, 4H), 3.51 (s, 2H), 2.86 (bs, 3H), 2.65 (s, 6H), 2.60-2.50 (m, 8H)

Proton signals at 4.19 and 3.51 ppm correspond to tartaric acid. Based on the integration of those peaks, tartaric acid to Compound 1 was consistently 2:1.

### Example 9A: Preparation of Crystalline Form A

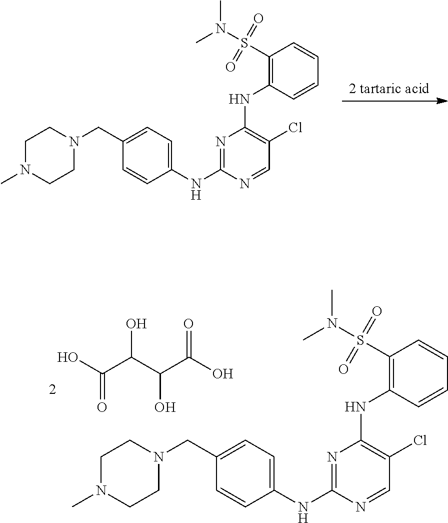

2-((5-chloro-2-((4-((4-methylpiperazin-1-yl)methyl)phenyl)amino)pyrimidin-4-yl)amino)-N,N-dimethylbenzenesulfonamide (5 g, 9.69 mmol) was dissolved in anisole (75 g) and EtOH (30 g) at 70° C. Tartaric acid (2.91 g, 19.38 mmol) dissolved in EtOH (30 g) was added to the mixture over 1 h, then small amount of seed crystal* was added to the solution to initiate the precipitation. The mixture was stirred for 1 h and cooled to 20° C. over 5 h. The solid was collected by filtration, washed with EtOH and dried to afford 2-((5-chloro-2-((4-((4-methylpiperazin-1-yl)methyl)phenyl)amino)pyrimidin-4-yl)amino)-N,N-dimethylbenzenesulfonamide di-tartrate salt as a white solid (7.35 g, 9.01 mmol).

The purity of the desired product was assessed to be 99.5% by HPLC.

¹H NMR (400 MHz, DMSO-d6) δ=9.56 (s, 1H), 9.33 (s, 1H), 8.58 (s, J=8.0 Hz, 1H), 8.29 (s, 1H), 7.83 (dd, J=8.0, 1.6 Hz, 1H), 7.71 (td, J=7.2, 1.4 Hz), 7.57 (d, J=8.4 Hz, 2H), 7.38 (td, J=7.2 Hz, J=1.0 Hz), 7.18 (d, J=8.4 Hz, 2H), 4.19 (s, 4H), 3.51 (s, 2H), 2.86 (bs, 3H), 2.65 (s, 6H), 2.60-2.50 (m, 8H)

Proton signals at 4.19 and 3.51 ppm correspond to tartaric acid. Based on the integration of those peaks, tartaric acid to Compound 1 was consistently 2:1.

* Note: Seed crystals of Form A were obtained by combining 2-((5-chloro-2-((4-((4-methylpiperazin-1-yl)methyl)phenyl)amino)pyrimidin-4-yl)amino)-N,N-dimethylbenzenesulfonamide and tartaric acid in anisole/ethanol under the conditions specified above, followed by initiation of crystal formation using one or more techniques such as (1) cooling the solution (e.g., to room temperature, or in a freezer), (2) concentrating the solution (e.g., by slow evaporation, or with a rotary evaporator), and/or (3) scratching the interior of the flask containing the solution. Crystals obtained in this manner were confirmed to be of Form A by ¹HNMR and XRPD analysis.

### Example 9B: Preparation of Crystalline Form B

2-((5-chloro-2-((4-((4-methylpiperazin-1-yl)methyl)phenyl)amino)pyrimidin-4-yl)amino)-N,N-dimethylbenzenesulfonamide mono-tartaric acid salt (1.75 kg, 2.62 mol) was added 5.25 L of EtOH and 4.0 L of solvent distilled under reduced pressure. This was repeated twice until the water content by Karl Fisher Titration is below 0.1%. Then 52.5 L of Ethanol and heated to reflux. Then 0.513 kg of L-(+)-tartaric acid in 17.5 L of ethanol was added slowly over 2 hours and seeded at 70° C. Then stirring was kept for 12 hours at 70° C. and again heated to 80° C. and kept for 2 hours. The slurry was cooled to 20° C. over 6 hours, and then kept stirring for additional 12 hours. The crystals were filtered and then washed with 3.5 L of Ethanol twice. The crystal was dried under reduced pressure to afford 1.8 kg of crystal. Then 1.6 kg of this material was suspended with 16.0 L of methyl t-butyl ether and 0.30 kg of bis(pinacolate) diboron was added at 30° C. Then the mixture was heated at 40-45° C. for 12 hours and cooled to 30° C. and filtered. The filtered solid was washed 8.2 L of methyl t-butyl ether and dried under vacuum to afford 2-((5-chloro-2-((4-((4-methylpiperazin-1-yl)methyl)phenyl)amino)pyrimidin-4-yl)amino)-N,N-dimethylbenzenesulfonamide mono-tartaric acid salt (1.38 kg, 2.07 mol).

### Example 9C: Preparation of Crystalline Form D

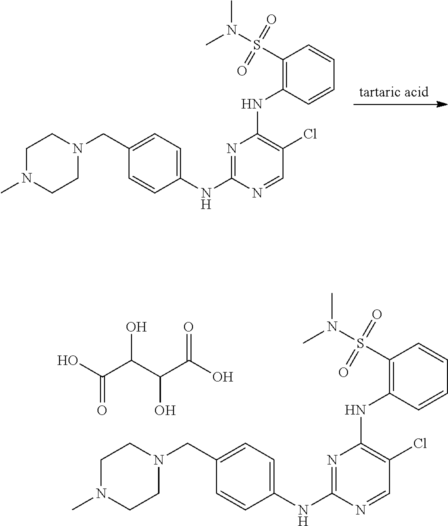

2-((5-chloro-2-((4-((4-methylpiperazin-1-yl)methyl)phenyl)amino)pyrimidin-4-yl)amino)-N,N-dimethylbenzenesulfonamide (5 g, 9.69 mmol) was charged into EtOH (119 g) and the mixture was heated to 80° C. Tartaric acid (1.45 g, 9.69 mmol) dissolved in EtOH (40 g) was added to the mixture over 2 h at the same temperature, then the mixture was cooled to 70° C. before small amount of seed crystal* was added to initiate the precipitation. The mixture was stirred for 2 h and cooled to 20° C. over 5 h. The solid was collected by filtration, washed with EtOH and dried to afford 2-((5-chloro-2-((4-((4-methylpiperazin-1-yl)methyl)phenyl)amino)pyrimidin-4-yl)amino)-N,N-dimethylbenzenesulfonamide mono-tartrate salt as a white solid (5.98 g, 8.98 mmol).

* Note: Seed crystals of Form D were obtained by combining 2-((5-chloro-2-((4-((4-methylpiperazin-1-yl)methyl)phenyl)amino)pyrimidin-4-yl)amino)-N,N-dimethylbenzenesulfonamide and tartaric acid in anisole/ethanol under the conditions specified above, followed by initiation of crystal formation using one or more techniques such as (1) cooling the solution (e.g., to room temperature, or in a freezer), (2) concentrating the solution (e.g., by slow evaporation, or with a rotary evaporator), and/or (3) scratching the interior of the flask containing the solution. Crystals obtained in this manner were confirmed to be of Form D by ¹HNMR and XRPD analysis.

### Example 10: Characterization of Various Polymorphic Forms of Tartrate Salts of Compound 1

### (i) Stability and Reproducibility

Other polymorphic forms of Compound 1 were compared for their relative stability under different storage conditions. Other polymorphic forms include the free base form of Compound 1, Form B, and Form D. The XRPD diffractogram of Forms B and D are shown in FIGS. 3 and 4, respectively.

It was discovered that Form A is a di-tartaric acid salt form and it was identified as having only one crystal form (Form A). For comparison, XPRD patterns showing an overlay of Form A (lower) and Form B (upper) illustrates a similar pattern, with some additional peaks.

### (ii) Stability Profiles

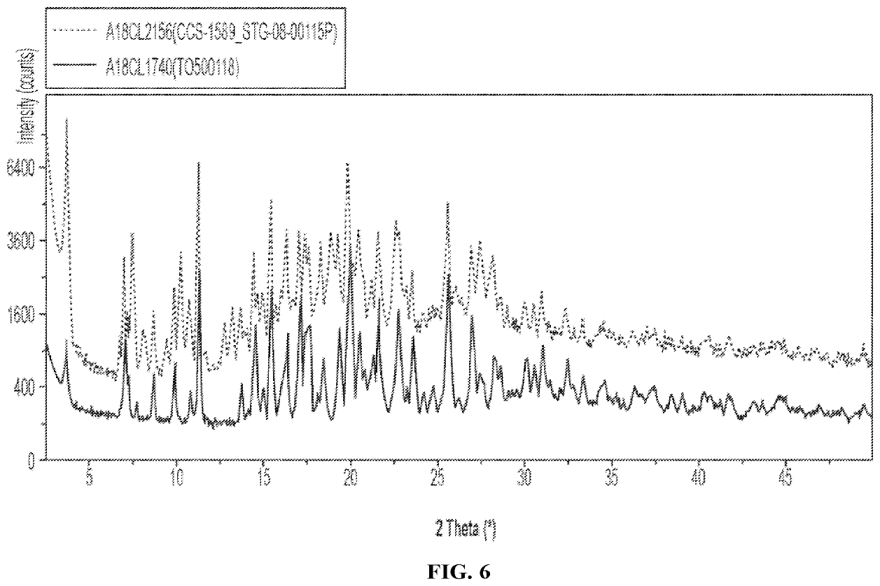

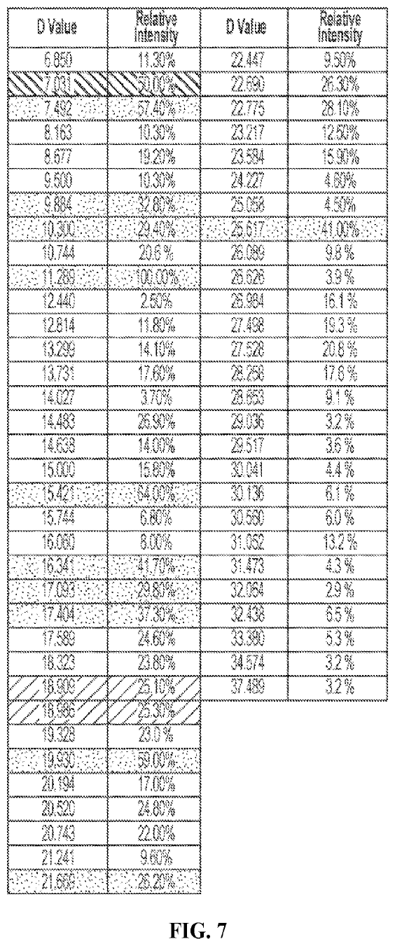

The different forms of Compound 1 (i.e., FB, Form A, Form B, and Form D were each stored at 40° C. and a relative humidity of 75%. After 3 weeks, all samples showed good chemical stability with no significant HPLC purity decrease. Form change was observed only for Form B. Additionally, as shown in FIG. 6, which compares Form A and Form B, Form B appears to have weaker peak intensities. The weaker peak intensity can possibly be attributed to lower crystallinity.

(iii) Physical and Chemical Properties

The table below summarizes the characterization and solid state stability results observed for FB and Forms A, B, and D. Samples were prepared and data was obtained according to the procedures and processes listed above unless otherwise specified. More procedures, processes, and results are listed below in Table 16.

TABLE 16
Summary of physicochemical properties evaluation for
Forms A, B, D, and free base

| Assay | | | | | |
|---|---|---|---|---|---|
| Description | | Form A | Form B | Form D | Free Base |
| XRPD | | Crystalline | Crystalline | Crystalline | Crystalline |
| Weight Loss (%) | | 1.8 (160° C.) | 2.3 (160° C.) | 2.0 (160° C.) | 0.22 (120° C.) |
| Endotherm (peak, ° C.) | | 107.8, | 101.9, 140.1 | 79.4, 140.7 | 221.1 |
| | | 152.1, 189.1 | | | |
| Initial HPLC Purity | | 99.6 | 98.1 | 98.8 | 98.3 |
| (area %) | | | | | |
| HPLC Purity at 3 weeks^{#} | | 99.7 | 98.2 | 98.3 | 98.6 |
| (area %) | | | | | |
| Form Change at 3 weeks^{#} | | No | Yes | No | No |
| Hygroscopicity/water | | 4.6%/No | 7.5%/No | 5.2%/Yes | Non-hygroscopic/ |
| uptake at 80% RH/FC | | | | | 0.07%/No |
| Morphology | | Fine particles with agglomeration | | | Diamond particles |
| Solubility* | SGF | >17.4/NA | >6.4/NA | >11.0/NA | 5.7/No |
| (mg/mL)/FC | FaSSIF | >14.8/NA | >8.9/NA | 3.9/Yes | 0.021/No |
| | FeSSIF | 5.2/NA* | 4.7/NA* | 4.6/NA* | 1.7/No |
| | pH 2.0 | >6.7/NA | >6.6/NA | >6.8/NA | 2.6/Yes |
| | pH 4.0 | 0.45/Yes | 0.26/Yes | 0.23/Yes | 0.17/Yes |
| | pH 6.0 | 3.8/Yes | 2.5/Yes | 1.9/Yes | 0.014/No |
| | pH 8.0 | 0.020/Yes | 0.010/Yes | 0.0050/Yes | 0.038/No |
| | pH 10.0 | 0.0098/Yes | 0.018/Yes | 0.053/Yes | 0.076/No |

*The solubility was collected at 24 hours
^{#}under 40° C./75% RH
FC: Form change
NA: solid-state results not available as clear solution formed
NA*: solid-state results not available as oil sample obtained.

(a) Dynamic Vapor Sorption (DVS)

DVS results showed up to 80% relative humidity at 25° C., the water uptake was in the range of 4.6˜7.5% for Forms A, B, and D with form change observed for Form D. That is, Form D changed to a new form not consistent with all the four known crystal forms, and free base was non-hygroscopic (water uptake less than 0.2%) with no form change at the end of the test.

(b) Kinetic Solubility

Kinetic solubility was measured in bio-relevant buffers (FaSSIF, FeSSIF and SGF) at 37° C. Compared to free base sample, significant higher solubility (>5 mg/mL) was observed for Forms A, B, and D in all biorelevant buffers. Similar solubility observations were observed for all three forms in SGF and FeSSIF, but Form D showed lower solubility than other Forms A and B in FaSSIF.

(c) pH solubility

pH solubility was measured in pH 2, 4, 6, 8 and 10 buffers at 37° C. The results showed: i) higher solubility in pH 2 than that of in the other pH buffers for each sample and decreased solubility in higher pH buffer, especially in alkaline media, in which the solid form of residual solids also changed at the end of the test for Forms A, B, and D; ii) Form A showed higher solubility than other polymorphs in pH 2, 4 and 6 buffers.

### Example 11: Solvent and Slurry Screening of Crystalline Forms

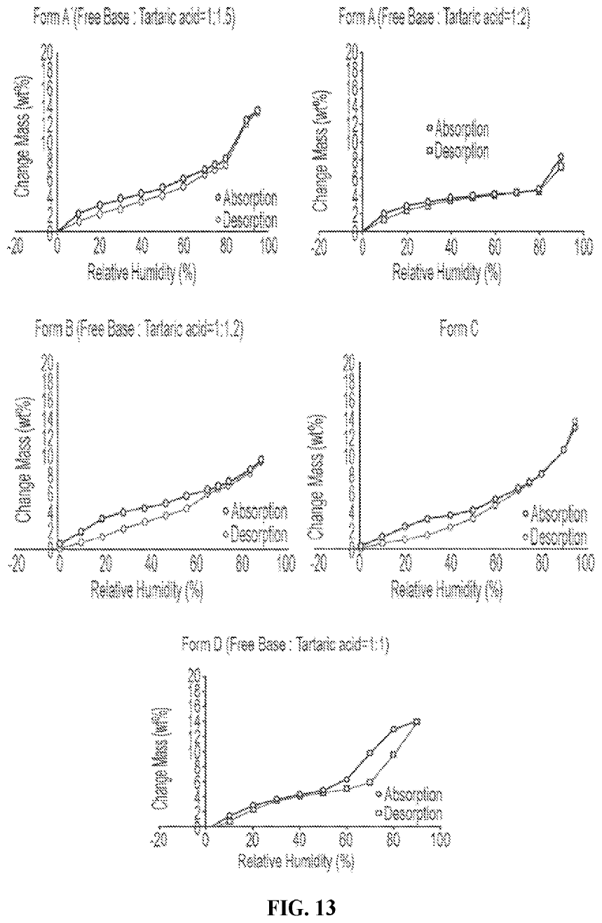

The experimental conditions and the brief results acquired from the solvent screening and the slurry screening are listed in Tables 3-6. The variable organic solvents and those mixtures with water were used as solvent systems. From these screening studies, ten crystal forms, Form A′, A, B, C, D, E, F, G, H and I, were assigned by XRPD patterns as shown in FIG. 11. The thermal behavior (DSC/TGA charts) for these crystals are also shown in FIGS. 12A-12I. The moisture sorption isotherms for Form A′, A, B, C, and D are provided in FIG. 13.

### Form A

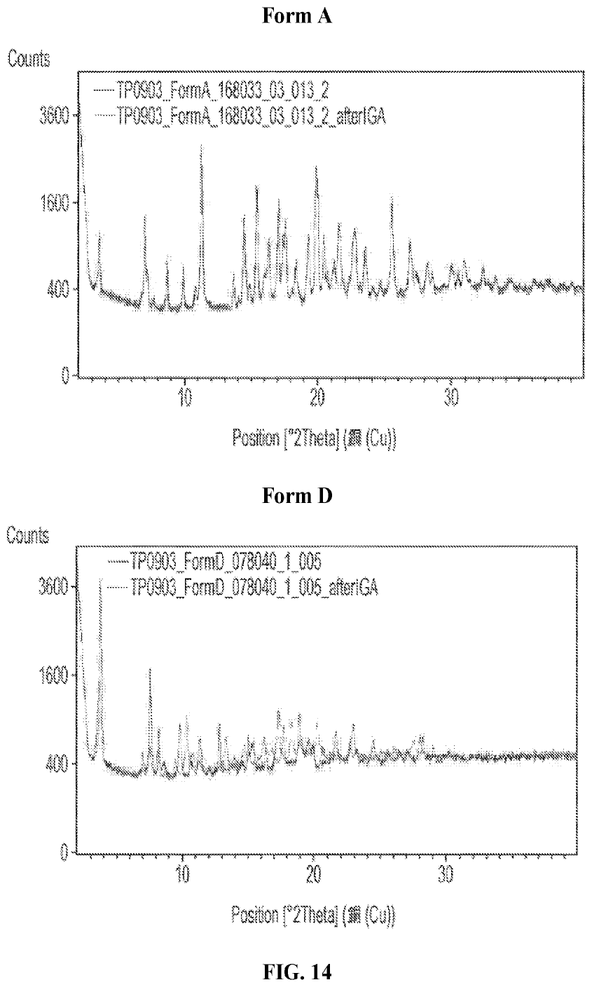

The solvent screen was conducted as shown in Table 4. Form A was not transformed and was recrystallized in almost the organic solvent systems without H₂O. Two types of the stoichiometric crystals, which were indicated from initial studies, were respectively used in the slurry screen as shown in Tables 3 and 5. They were maintained in almost the solvent systems except for H₂O during the screen. This indicated that Form A was basically stable and dominant form. Form A was transformed to Form B in H₂O, like as that observed in the solvent screen. The thermal analysis charts for the two types of Form A were shown in FIG. 99A. The significant weight-loss were observed for these batches with the higher melting point than that expected. The moisture sorption isotherm showed that these Form A were hygroscopic as shown in FIG. 13, and the additional XRPD analysis as shown in FIG. 14 found that Form A was maintained after the moisture absorption/desorption cycle. No significant changes were observed during the stability studies as shown in Table 17.

TABLE 17
Summary of Stability Results

| Form | Initial | 40° C. | 40° C./75% RH | 60° C. | 60° C./75% RH |
|---|---|---|---|---|---|
| A′ | 97.9% | 98.1% | 98.0% | 97.8% | 98.1% |
| A | 99.3% | 99.3% | 99.3% | 99.3% | 99.3% |
| D | 98.4% | 98.2% | 98.4% | 98.2% | 98.2% |

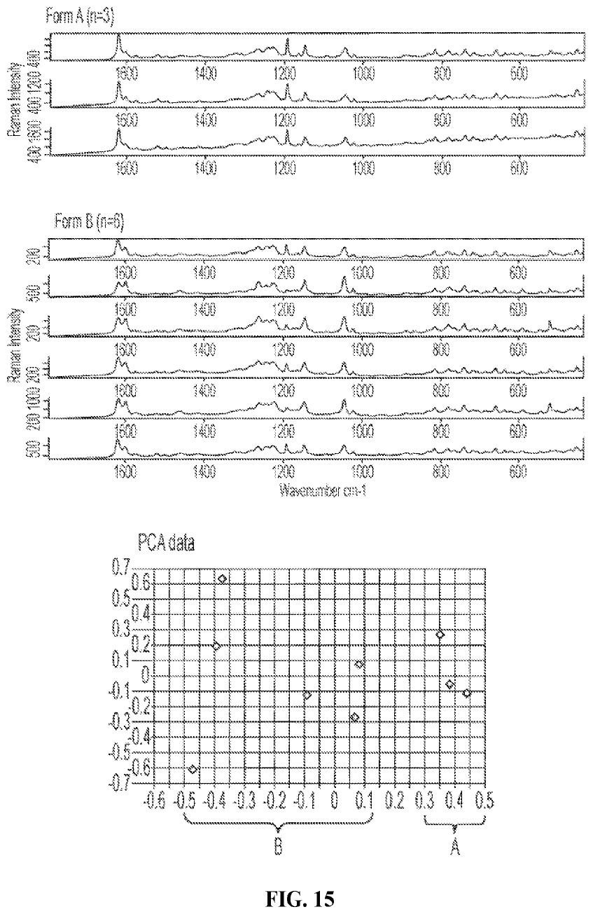

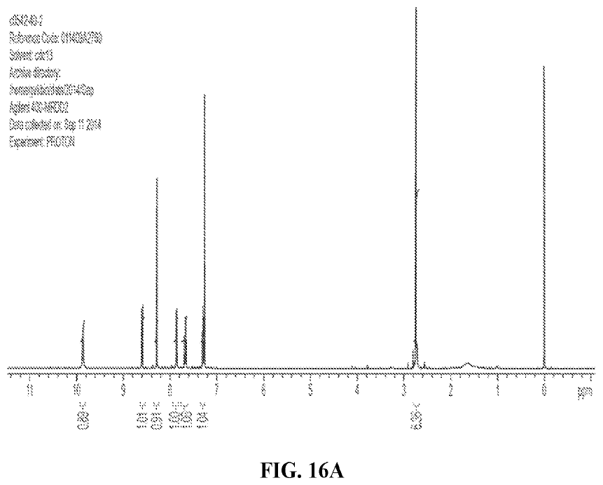

The two types of the stoichiometric crystals of Form A were finally identified since the XRPD pattern of the 2 batches of Compound 1 tartrate salt were same but the quantitative analyses by ion chromatography were different. The ratios of free base and tartaric acid for these two types of stoichiometric crystal were 1:1.5 (Form A′) and 1:2 (Form A). The Raman spectra of Form A were consistent as shown in FIG. 15.

### Form B

In the solvent and slurry screen, Form A was transformed to Form B by being recrystallized/suspended in H₂O, as shown in Table 4 and Table 5. The XRPD pattern of Form B was similar to that of Form A but it seemed to be contaminated with another crystal form as shown in FIG. 98. About 4%-weight loss and a broad endotherm peak derived from adhered water/solvent was observed and the melting point was over 130° C. as shown in FIG. 12B. The moisture sorption isotherm showed that Form B was hygroscopic as shown in FIG. 13. The quantitative analysis by ion chromatography indicated that the stoichiometry of freebase and tartaric acid was 1:1.2. The representative Raman spectrum was shown in FIG. 15, but the Raman spectra varied depending on the measured area of the material. This result strongly suggested that Form B was mixture of Form A and another form.

### Form C

In the solvent screen, as shown in Table 4, Form C was formed by the recrystallization of Form A with the mixture of H₂O and alcohol, such as methanol and 2-propanol. About 10%-weight loss and broad endotherm peak was observed in the thermal analysis chart as shown in FIG. 12C. That suggested that Form C might be a solvate with alcohol and the alcohol was eliminated depending on the increase of temperature. The moisture sorption isotherm as shown in FIG. 13 indicated that Form C was hygroscopic.

### Form D

Form D was not formed in the solvent screen from Form A. Form D used in the slurry screen was not transformed in ethanol and 2-propanol, but done to various forms, E, F, G, H and I, in the other solvents, as shown in Table 6. This data indicated that Form D would be difficult to control in the manufacturing process. About 3%-weight loss and a broad endotherm peak due to adhered water/solvent was observed in the thermal analysis chart as shown in FIG. 12D. The moisture sorption isotherm as shown in FIG. 13 indicated that Form D was hygroscopic. The additional XRPD analysis as shown in FIG. 14 found that Form D was transformed to the other forms after the moisture absorption/desorption cycle. No significant changes were observed during the stability studies as shown in Table 19. The stoichiometry of freebase and tartaric acid is 1:1.

### Form E

Form E was formed in the slurry screen from Form D only in methanol at room temperature, as shown in Table 6. About 4%-weight loss was observed in the thermal analysis chart as shown in FIG. 12E.

### Form F

Form F was formed in the slurry screen from Form D in the mixture of alcohol and H₂O at room temperature and 50° C. as shown in Table 6. About 6%-weight loss was observed in the thermal analysis chart as shown in FIG. 12F.

### Form G

Form G was formed in the slurry screen from Form D in H₂O at room temperature and 50° C. as shown in Table 6. The thermal analysis chart was provided in FIG. 12G. No further studies could be done because of the insufficient amount of sample.

### Form H

Form H was formed in the slurry screen from Form D only in Methanol-H₂O (5:1) at room temperature, as shown in Table 6. The thermal analysis chart is provided in FIG. 12H.

### Form I

Form I was formed in the slurry screen from Form D only in acetnimile-H₂O (10:1) at 50° C., as shown in Table 6. Thermal analysis data is provided in FIG. 12I.

### CONCLUSION

The polymorph screen revealed that Compound 1 tartrate salt had the nine crystal forms involving solvates. Among them, Form A could be the most suitable form for the tartrate salt when its stoichiometry is well-controlled in the manufacturing process, in terms of the dominance in the screens and the acceptable solid-form properties.

Although the disclosure has been described with reference to a specific embodiment this description is not meant to be construed in a limiting sense. The disclosure being thus described, it is apparent that the same can be varied in many ways. Such variations are not to be regarded as a departure from the spirit and scope of the present disclosure, and all such modifications, alternatives, and equivalents as would be obvious to one skilled in the art are intended to be included within the scope of the following claims.

We claim:

1. A formulation comprising:
Compound 1 or a pharmaceutically acceptable salt thereof; 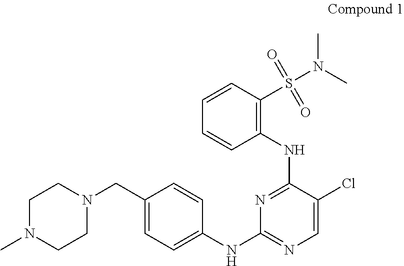
microcrystalline cellulose;
lactose monohydrate;
croscarmellose sodium; and
magnesium stearate.

2. The formulation of claim 1, wherein the Compound 1 or a pharmaceutically acceptable salt thereof is a tartrate salt of Compound 1.

3. The formulation of claim 2, wherein the tartrate salt of Compound 1 is a di-tartrate salt of Compound 1.

4. The formulation of claim 2, wherein the tartrate salt of Compound 1 is a mono-tartrate salt of Compound 1.

5. The formulation of claim 2, wherein the tartrate salt of Compound 1 is a sub-tartrate salt of Compound 1.

6. The formulation of claim 3, wherein the di-tartrate salt of Compound 1 is a crystalline salt of Form A.

7. The formulation of claim 4, wherein the mono-tartrate salt of Compound 1 is a crystalline salt of Form D.

8. The formulation of claim 5, wherein the sub-tartrate salt of Compound 1 is a crystalline salt of Form B.

9. The formulation of any one of claims 1-8, comprising:
from about 0.5 wt % to about 30 wt % of tartrate salt of Compound 1;
from about 10 wt % to about 30 wt % microcrystalline cellulose;
from about 50 wt % to about 80 wt % lactose monohydrate;
from about 1 wt % to about 5 wt % croscarmellose sodium; and
from about 0.5 wt % to about 2 wt % magnesium stearate.

10. The formulation of claim 1, 3, or 9, wherein the formulation comprises a unit dosage of crystalline Form A of the di-tartrate salt of Compound 1 of 4 mg, 25 mg, or 100 mg.

11. The formulation of any one of claims 1-10, wherein the formulation is a dry blended powder.

12. The formulation of any one of claims 1-11, wherein the formulation is a capsule.

13. The formulation of claim 9, wherein the formulation is a capsule, and wherein the percentages of tartrate salt of Compound 1, microcrystalline cellulose, lactose monohydrate, croscarmellose sodium, and magnesium stearate are expressed in weight percent of the capsule exclusive of capsule shell.

14. A formulation comprising
crystalline Form A of a di-tartrate salt of Compound 1;
microcrystalline cellulose;
lactose monohydrate;
croscarmellose sodium; and
magnesium stearate.

15. A formulation comprising:
from about 0.5 wt % to about 30 wt % of crystalline Form A of a di-tartrate salt of Compound 1;
from about 10 wt % to about 30 wt % microcrystalline cellulose;
from about 50 wt % to about 80 wt % lactose monohydrate;
from about 1 wt % to about 5 wt % croscarmellose sodium; and
from about 0.5 wt % to about 2 wt % magnesium stearate.

16. A unit dosage form comprising:
about 4 mg of crystalline Form A of a di-tartrate salt of Compound 1;
from about 30 to about 40 mg of microcrystalline cellulose;
from about 100 to about 150 mg of lactose monohydrate;
from about 4 to about 6 mg of croscarmellose sodium; and
from about 1 to about 2.5 mg of magnesium stearate.

17. A unit dosage form comprising:
about 25 mg of crystalline Form A of a di-tartrate salt of Compound 1;
from about 27 to about 37 mg of microcrystalline cellulose;
from about 80 to about 120 mg of lactose monohydrate;
from about 4 to about 6 mg of croscarmellose sodium; and
from about 1 to about 2.5 mg of magnesium stearate.

18. A unit dosage form comprising:
about 100 mg of crystalline Form A of a di-tartrate salt of Compound 1;
from about 50 to about 70 mg of microcrystalline cellulose;
from about 150 to about 250 mg of lactose monohydrate;
from about 8 to about 14 mg of croscarmellose sodium; and
from about 2 to about 5 mg of magnesium stearate.

19. A capsule comprising a dry blended powder enclosed within a capsule shell, the dry blended powder comprising:
tartrate salt of Compound 1;
microcrystalline cellulose;
lactose monohydrate;
croscarmellose sodium; and
magnesium stearate.

20. The capsule of claim 19, wherein the tartrate salt of Compound 1 is a di-tartrate salt of Compound 1.

21. The capsule of claim 19, wherein the tartrate salt of Compound 1 is a mono-tartrate salt of Compound 1.

22. The capsule of claim 19, wherein the tartrate salt of Compound 1 is a sub-tartrate salt of Compound 1.

23. The capsule of claim 20, wherein the di-tartrate salt of Compound 1 is a crystalline salt of Form A.

24. The capsule of claim 21, wherein the mono-tartrate salt of Compound 1 is a crystalline salt of Form D.

25. The capsule of claim 22, wherein the sub-tartrate salt of Compound 1 is a crystalline salt of Form B.

26. The capsule of any one of claims 19-25, wherein the dry blended powder comprises:
from about 0.5 wt % to about 30 wt % of tartrate salt of Compound 1;
from about 10 wt % to about 30 wt % microcrystalline cellulose;
from about 50 wt % to about 80 wt % lactose monohydrate;
from about 1 wt % to about 5 wt % croscarmellose sodium; and
from about 0.5 wt % to about 2 wt % magnesium stearate.

27. The capsule of any one of claims 19, 20, 23, or 26, wherein the capsule comprises a unit dosage of crystalline Form A of the di-tartrate salt of Compound 1 of 4 mg, 25 mg, or 100 mg.

28. A process for preparing a capsule formulation, comprising:
sifting Compound 1 or a pharmaceutically acceptable salt thereof;
sifting lactose monohydrate;
blending the Compound 1 or a pharmaceutically acceptable salt thereof and lactose monohydrate with microcrystalline cellulose to form a first dry blend;
blending the first dry blend with croscarmellose sodium to form a second dry blend;
blending magnesium stearate with the second dry blend to form a final dry blend; and
filling a capsule shell with the final dry blend to form the capsule formulation.

29. The process of claim 28, wherein the lactose monohydrate is blended gradually with the microcrystalline cellulose and Compound 1 or a pharmaceutically acceptable salt thereof.

30. The process of claim 28 or 29, wherein blending to form the first dry blend and/or second dry blend comprises blending at from about 40 to about 45 rpm.

31. A process for preparing a capsule formulation, comprising:
triturating Compound 1 or a pharmaceutically acceptable salt thereof and lactose monohydrate in a mortar to form a triturate;
blending the triturate with microcrystalline cellulose in a blender to form a first dry blend;
blending the first dry blend with croscarmellose sodium to form a second dry blend;
blending magnesium stearate with the second dry blend to form a final dry blend; and
filling a capsule shell with the final dry blend to form the capsule formulation.

32. The process of claim 31, wherein the lactose monohydrate is added gradually during trituration.

33. The process of claim 31 or 32, comprising sifting Compound 1 or a pharmaceutically acceptable salt thereof and/or lactose monohydrate prior to trituration.

34. The process of any one of claims 31-33, comprising sifting the microcrystalline cellulose, croscarmellose sodium, and/or magnesium stearate prior to blending.

35. The process of any one of claims 31-34, wherein blending to form the first dry blend and/or the second dry blend comprises blending at from about 40 to about 45 rpm.

36. The process of any one of claims 31-35, wherein the Compound 1 or a pharmaceutically acceptable salt thereof is a tartrate salt.

37. The process of claim 36, wherein the tartrate salt of Compound 1 is a di-tartrate salt of Compound 1.

38. The process of claim 36, wherein the tartrate salt of Compound 1 is a mono-tartrate salt of Compound 1.

39. The process of claim 36, wherein the tartrate salt of Compound 1 is a sub-tartrate salt of Compound 1.

40. The process of claim 37, wherein the di-tartrate salt of Compound 1 is a crystalline salt of Form A.

41. The process of claim 38, wherein the mono-tartrate salt of Compound 1 is a crystalline salt of Form D.

42. The process of claim 39, wherein the sub-tartrate salt of Compound 1 is a crystalline salt of Form B.

43. The process of any one of claims 28-42, wherein the final dry blend comprises:
from about 0.5 wt % to about 30 wt % of tartrate salt of Compound 1;
from about 10 wt % to about 30 wt % microcrystalline cellulose;
from about 50 wt % to about 80 wt % lactose monohydrate;
from about 1 wt % to about 5 wt % croscarmellose sodium; and
from about 0.5 wt % to about 2 wt % magnesium stearate.

44. A formulation prepared in accordance with the process of any one of claims 28-43 having the weight percentage of components shown in the following table:

| Component | |
|---|---|
| Crystalline Form B of a sub-tartrate salt of Compound 1 | 0.588 wt % |
| Microcrystalline cellulose | 22.0 wt % |
| Lactose Monohydrate | 73.4 wt % |
| Croscarmellose Sodium | 3.0 wt % |
| Magnesium Stearate | 1.0 wt %. |

45. A formulation prepared in accordance with the process of any one of claims 28-43 having the weight percentage of components shown in the following table:

| Component | |
|---|---|
| Crystalline Form B of a sub-tartrate salt of Compound 1 | 2.35 wt % |
| Microcrystalline cellulose | 21.65 wt % |
| Lactose Monohydrate | 72.0 wt % |
| Croscarmellose Sodium | 3.0 wt % |
| Magnesium Stearate | 1.0 wt %. |

46. A formulation prepared in accordance with the process of any one of claims 28-43 having the weight percentage of components shown in the following table:

| Component | |
|---|---|
| Crystalline Form B of a sub-tartrate salt of Compound 1 | 9.41 wt % |
| Microcrystalline cellulose | 20.24 wt % |
| Lactose Monohydrate | 66.35 wt % |
| Croscarmellose Sodium | 3.0 wt % |
| Magnesium Stearate | 1.0 wt. %. |

47. A formulation prepared in accordance with the process of any one of claims 28-43 having the weight percentage of components shown in the following table:

| Component | |
|---|---|
| Crystalline Form A of a di-tartrate salt of Compound 1 | 2.35 wt % |
| Microcrystalline cellulose | 21.64 wt % |
| Lactose Monohydrate | 72.0 wt % |
| Croscarmellose Sodium | 3.0 wt % |
| Magnesium Stearate | 1.0 wt %. |

48. A formulation prepared in accordance with the process of any one of claims 28-43 having the weight percentage of components shown in the following table:

| Component | |
|---|---|
| Crystalline Form A of a di-tartrate salt of Compound 1 | 14.7 wt % |
| Microcrystalline cellulose | 18.9 wt % |
| Lactose Monohydrate | 62.4 wt % |
| Croscarmellose Sodium | 3.0 wt % |
| Magnesium Stearate | 1.0 wt %. |

49. A formulation prepared in accordance with the process of any one of claims 28-43 having the weight percentage of components shown in the following table:

| Component | |
|---|---|
| Crystalline Form A of a di-tartrate salt of Compound 1 | 26.32 wt % |
| Microcrystalline cellulose | 16.05 wt % |
| Lactose Monohydrate | 53.63 wt % |
| Croscarmellose Sodium | 3.0 wt % |
| Magnesium Stearate | 1 wt %. |
